(19) United States
(12) Patent Application Publication
(10) Pub. No.: US 2023/0390463 A1
(43) Pub. Date: Dec. 7, 2023

(54) TISSUE SCAFFOLDS

(71) Applicant: JD BIOMEDICAL SOLUTIONS LTD., Oxfordshire (GB)

(72) Inventors: Catriona Bethan INVERARITY, York (GB); Julian Francis DYE, Oxfordshire (GB)

(21) Appl. No.: 18/250,873

(22) PCT Filed: Oct. 27, 2020
(86) PCT No.: PCT/EP2021/079887

§ 371 Date: Apr. 27, 2023

(30) Foreign Application Priority Data
Oct. 27, 2020 (GB) 2017052.8

(51) Int. Cl. A61L 27/60; A61L 27/22; C12N 5/00

(52) U.S. Cl. CPC A61L 27/60; A61L 27/225; C12N 5/0068; C12N 2537/10; C12N 2533/56; C12N 2533/30; C12N 2513/00

(57) ABSTRACT

The present invention relates to a protein-based three-dimensional tissue scaffold and the method of the manufacture thereof. Instant scaffolds are particularly useful in tissue reconstruction, in particular in wound treatment and as dermal replacement scaffolds.

### FIELD OF THE INVENTION

The present invention relates to three-dimensional protein-based tissue scaffolds, methods of their manufacture and their applications. Instant scaffolds are useful in therapy, in particular in tissue replacement and wound treatment. Additionally, micronized forms can be injected through a cannula to fill crevices, fistulae intra-cutaeous or sub-cutaneous tracts.

### BACKGROUND OF THE INVENTION

Chronic wounds, such as venous ulcers and diabetic ulcers, affect a significant proportion of the population. In the UK alone, the annual incidence of patient receiving treatment for chronic wounds is estimated at 4.5 million people in 2020 and cost the NHS an estimated £3bn a year. There are an estimated 24,000 UK hospital admissions for diabetic foot ulcers alone. With National Institute for Health and Care Excellence estimating the number of people living with diabetes to almost double between 2013 and 2025, this figure is only expected to increase. Amid growing pressures on health services and care provision, there is a requirement to move towards treatment, rather than management, of chronic wounds. This would not only reduce the economic burden of such wounds, but also more importantly improve patient outcome and quality of life.

Biomaterials have long been investigated as a means of supporting regeneration of tissue damaged by burns or chronic conditions. Porous synthetic acellular scaffolds formed from biomaterials such as blended collagen and chondroitin sulphate, collagen and elastin, or esterified hyaluronan have been established as safe and feasible materials to support cell ingrowth and tissue formation within the matrix of the synthetic structure. Although such materials have become clinically established, their use in practice is limited by factors such as wound infection, slow rate of blood capillary ingrowth, rapidity of resorption or conversely excessive stability in the wound.

Moreover, cell therapies and in vitro tissue-engineered skin equivalents, for wound healing are also well established, and also have limited clinical efficacy, with poor integration, and limited grafts duration, and inadequate dermal reconstruction and scarring. The use of scaffolds for supporting allogenic cells has been under development for the last 25 years; however, few integrated solutions with suitable scaffolds and have been developed. Additionally, few scaffold solutions are being produced and marketed at the scale, cost and speed required to seriously tackle the growing burden of chronic wounds.

A scaffold is a substrate material into which cells may grow into from surround tissues, or it may be loaded for transplantation. Much like building scaffolding, it creates a supportive structural framework which plugs a physical gap in the injured tissue, while providing an internal microenvironment that houses cells (such as stem cells), allowing them to communicate, produce extracellular matrix and ultimately regenerate the wound by producing healthy tissue that integrates into its surroundings. The scaffolding material itself may promote wound healing by direct interaction with ingressing cells, or by release of bioactive agents. This may be achieved by selection of a compatible, bioactive material such as a native protein, or an inert substrate functionalised with a bioactive coating.

In the last century there has been enormous development in the treatment of serious skin injuries, particularly with the advent of modern tissue engineering. However, there has always been a lag between laboratory developments and improved clinical outcomes. Historically this has been due to lack of detailed knowledge of the pathophysiology in these complex wound environments. More recent challenges include how to promote regeneration (newly formed matrix resembling native tissue, with total function and no scarring) as opposed to loss of tissue or healing with scarring, contracture or other loss of function.

When the epidermis is breached in a superficial wound, keratinocytes migrate from the wound periphery and bed, leading to total regeneration with no visible scar. For larger epidermal defects, keratinocyte migration from the wound margins alone is not sufficient to cover the wound and restore the epidermal barrier. A single sheet layer of cultured keratinocytes can be used to replace irreparably damaged epidermis. However, in deeper wounds that extend into the dermis, the dermal structures that support healing are compromised and tissue will healing by secondary intention and form scar tissue, rather than regenerate. The dermis is not capable of regeneration so this process of scarring is insufficient to adequately replace a full-thickness defect, either cosmetically or functionally. To replicate the thickness, durability and difference in both anatomical and mechanical structures, a second ‘dermal’ layer is required.

Early synthetic scaffolds sought to restore epidermal barrier function by creating a seal to prevent infection and fluid loss. The materials used were typically bioinert organic polymers which adhered to the wound and provided no stimulus to influence or promote wound healing. The recognition of biocompatible and bioactive materials led to the creation of more ambitious constructs. These aimed not only to replace missing epidermis, but also to support the damaged dermis by provision of a bilayered construct. This tenet provided the basis for Integra® (Integra LifeSciences; Plainsboro, NJ, USA); comprising a temporary Silastic outer layer and a porous collagen-chondroitin ‘dermis’. The silicon ‘epidermal’ layer is initially bonded to the ‘dermis’ once the porous dermal component has integrated with host tissue, the Silastic may be peeled away, allowing the epidermis to heal from the wound margin and burgeoning dermal wound bed, or by the application of autograft. The porous collagen-chondroitin scaffold supports in-migration of cells from the surrounding dermis, and was found to vascularise within 3 to 5 days of implantation. Integra® was an important development in the treatment of burn wounds, arguably the most significant since the Second World War, as it represented a shift in thinking from merely sealing the wound to reduce mortality, to healing the wound and restoring appearance and functionality

More recently, it has been shown that cultured autologous keratinocytes and fibroblasts, seeded onto a collagen-based sponge could be used in conjunction with Integra® in place of an autograft. This approach requires a biopsy to harvest autologous cells, but removing the necessity for repeated autograft harvests. If the biopsy is taken early, the cells may be sufficiently expanded to apply when the Silastic layer is ready for removal. There is no need to wait for re-epithelialisation of graft sites to harvest autografts, and the smaller area and duration of exposed tissue from biopsy reduces the risk of infection.

MySkin (Regenerys, Cottenham, Cambridge, UK) also uses the concept of in vitro expansion of autologous keratinocytes to avoid the need for extensive and repeated autograft harvest in burns and chronic wounds patients. Traditionally taking a similar form to the cultured skin substitute described previously, keratinocytes are expanded in vitro and delivered to the wound on a polymer carrier dressing, allowing a convenient patch method of applying cells to the wound. MySkin is also available as a spray, where keratinocytes are delivered in a suspension and may be applied by spraying directly onto the wound bed.

Dermal scaffolds are designed to support cells that will mimic the native dermis. The dermis is highly vascularised; it supports the keratinised avascular epidermis by nutrient exchange by diffusion. As such, a key feature of dermal scaffolds—as opposed to typical dressings or epidermal scaffolds—is the ability to induce or encourage angiogenesis into the grafted scaffold. This will support the regenerating tissue and allow the damaged dermis to continue to support the regenerating epidermis above. Dermal scaffolds may be optimised to enhance angiogenic potential by modifying the size, number and interconnectivity of pores; using bioactive materials, releasing agents or coatings to stimulate angiogenesis; or by chemically modifying the scaffold to present a more biocompatible surface to the wound margin.

Skin substitutes have been commercially available since the advent of modern tissue engineering in the 1970s. This began with the pioneering of autograft techniques, providing a rapid wound covering without the need for donor screening or the risk of immune rejection. However, the procedure is painful and may cause donor site morbidity and exposes additional surface area to infection in an already compromised patient. Autograft is therefore unsuited to treatment of extensive injuries. The use of artificial skin overcomes the drawbacks of the use of autograft and allograft/xenograft. There is no donor site morbidity and no wait for donor recruitment and screening. Synthetic products can be engineered to render them non-immunogenic. A range of products, for treatment of both chronic wounds and burns, is now commercially available for therapeutic use in patients where autograft is not a tenable solution.
Patent application US 2009/017092 relates to certain artificial extra cellular matrix product and artificial human and animal tissue product comprising tissue specific cells.
Andrea Barbetta et al. (Biomacromolecules, 2006, doi: 10.1021/bm0605331) discloses certain PolyHIPEs and their performance as scaffolds in the culture of hepatocytes.
M. de Colli et al. (Biomedical Materials, 2012, doi: 10.1088/1784-6041/7/5/055005) discloses certain biomimetic porous hydrogen and its application in the culture of hepatocytes.
Patent application WO 2013/164635 discloses a process for preparing a certain extracellular matrix—a synthetic skin scaffold.
Lim Xuxin et al. (Journal of Material Science, 2018, doi: 10.1007/S10856-018-6060-6) discloses manufacture and characterization of certain scaffolds for tissue regeneration.

### SUMMARY OF THE INVENTION

Skin is the largest organ of the body and is instrumental in homeostatic regulation as well as forming a physical barrier to infection. When skin is wounded and this protective barrier is breached, it is important to repair the damage quickly to restore function. However, some wounds do not heal readily, leaving patients in pain and at risk of secondary illnesses. These non-healing wounds may be chronic wounds such as ulcers, but another example is extensive partial- or full-thickness burns. The normal wound healing mechanism may not be impaired in the same way that it is in chronic wounds, but the extent of severe burn injuries means that the time required to restore the epidermal layer is excessively long. There may also be insufficient healthy tissue remaining for graft repair.

Cell therapies are typically limited by the rapid dispersal of cells on delivery. Therefore, scaffolds offer an approach of enhancing therapeutic efficiency and efficacy. The scaffold must be capable of physically filling the defect caused by the tissue damage (or the excised wound site) and integrating with the healthy surrounding tissue. This may be achieved by either ingress of migratory cells and migration of wound margin keratinocytes, or cellular colonisation of the scaffold before implantation; or a combination of the two. The scaffold must have a high degree of porosity with good interconnectivity to allow for the movement of cells and diffusion of nutrients and waste. Pores must be of an appropriate size to host cells and allow them to develop an appropriate morphology and proliferate.

The problem of providing a scaffold with desired properties is solved by the embodiments described herein and as characterized in the claims.

The present inventors have surprisingly found that a porous three-dimensional protein-based biomaterial scaffold is obtainable by using an emulsion templating technique using High Internal-Phase Emulsion (HIPE). Tem plating enables reproducible manufacture of materials with a regular, highly porous structure with a high degree of pore interconnectivity and consistent pore size distribution. The present inventors have further established the conditions for obtaining the high-internal phase emulsion, defining the surfactants that are useful in stabilizing the HIPE. According to the methods of the present invention, a range of pore diameters suitable for skin tissue engineering could be obtained. The resulting scaffold is suited to cell ingress and supporting angiogenesis and allows fluid and nutrient exchange between the scaffold and the wound environment.

The invention will be summarized in the following embodiments.

In a first embodiment, the present invention relates to a method for manufacturing a protein-based three-dimensional tissue scaffold, the method comprising the steps of: (a) providing at least one protein capable of triggered polymerization; (b) forming a high-internal phase oil/water emulsion comprising the protein(s) of step (a) in aqueous phase, further comprising a surfactant which supports a high-internal phase emulsion regime; (c) triggering polymerization of the at least one protein comprised in the high-internal phase emulsion of step (b); and (d) washing out the oil phase.

In a particular embodiment, the present invention relates to a method for manufacturing a protein-based three-dimensional tissue scaffold, wherein the at least one protein capable of triggered polymerization of step (a) is selected from fibrinogen, collagen, laminin, elastin, cultured cell extracellular matrix extracts, and extracellular matrix preparations from natural tissue.

In a further particular embodiment, the present invention relates to a method for manufacturing a protein-based three-dimensional tissue scaffold, wherein the at least one protein capable of triggering polymerization of step (a) is fibrinogen, and wherein step (c) is performed by addition of thrombin to the emulsion of step (b).

In again a further particular embodiment, the present invention relates to a method for manufacturing a protein-based three-dimensional tissue scaffold, wherein the surfactant which supports the high-internal phase emulsion regime of step (b) comprises: at least one optionally substituted polyethylene glycol chain comprising 5 to 16 ether bonds, and at least one aliphatic hydrocarbon chain of at least 8 carbon atoms, wherein at least one tertiary or quaternary carbon atom is present in the at least one aliphatic hydrocarbon chain.

In again a further particular embodiment, the present invention relates to a method for manufacturing a protein-based three-dimensional tissue scaffold, wherein the surfactant which supports the high-internal phase emulsion regime of step (b) exhibits oil-carrying capacity (OCC) in the range from 75% to 95% and preferably hydrophilic-lipophilic balance (HLB) in the range from 12 to 16.

In again a further particular embodiment, the present invention relates to a method for manufacturing a protein-based three-dimensional tissue scaffold, wherein the surfactant which supports the high-internal phase emulsion regime of step (b) comprises non-ionic surfactant and surfactants, with OCC in the range from 75% to 95% and preferably hydrophilic-lipophilic balance (HLB) in the range from 12 to 16, wherein ionic surfactant constitutes not more than 10 molar % of the total surfactant amount.

In again a further particular embodiment, the present invention relates to a method for manufacturing a protein-based three-dimensional tissue scaffold, wherein the surfactant which supports the high-internal phase emulsion regime of step (b) is selected from tergitol-NP10, Tergitol-TMN-6; Tergitol-TMN-10, triton-X100-R, Triton X104R, Triton X114R, Triton-CG110, Triton X165, and Ecosurf-EH-9.

In again a further particular embodiment, the present invention relates to a method for manufacturing a protein-based three-dimensional tissue scaffold, further comprising a step of crosslinking the protein-based three-dimensional tissue scaffold obtained in step (d).

In again a further particular embodiment, the present invention relates to a method for manufacturing a protein-based three-dimensional tissue scaffold, further comprising a step of freeze-drying the protein-based three-dimensional tissue scaffold.

In a further embodiment, the present invention relates to a protein-based three-dimensional tissue scaffold obtainable according to the method for manufacturing a protein-based three-dimensional tissue scaffold of the present invention.

In a particular embodiment, the present invention relates to a protein-based three-dimensional tissue scaffold obtainable according to the method for manufacturing a protein-based three-dimensional tissue scaffold of the present invention, comprising polymerized protein(s) of porosity of at least 74%.

In a further particular embodiment, the present invention relates to a protein-based three-dimensional tissue scaffold obtainable according to the method for manufacturing a protein-based three-dimensional tissue scaffold of the present invention, wherein the polymerized protein(s) comprise(s) fibrin.

In again a further particular embodiment, the present invention relates to a protein-based three-dimensional tissue scaffold obtainable according to the method for manufacturing a protein-based three-dimensional tissue scaffold of the present invention, further comprising a non-polypeptide polymer.

In a further embodiment, the present invention relates to a protein-based scaffold obtainable according to the method for manufacturing a protein-based three-dimensional tissue scaffold of the present invention, comprising fibrin and polyvinylalcohol.

In a further embodiment, the present invention relates to a method of producing a cell culture substrate, comprising a step wherein the protein-based three-dimensional tissue scaffold of the present invention is deposited using additive manufacturing techniques.

In again a further embodiment, the present invention relates to a use of the protein-based three-dimensional tissue scaffold of the present invention in an in vitro tissue culture.

In again a further embodiment, the present invention relates to a use of the protein-based three-dimensional tissue scaffold of the present invention in an in vitro tissue engineering.

In a particular embodiment, the present invention relates to a use of the protein-based three-dimensional tissue scaffold of the present invention in an in vitro tissue engineering, wherein in vitro tissue engineering comprises preparation of a myocardial patch.

In a further embodiment, the present invention relates to the protein-based three-dimensional tissue scaffold of the present invention for use in therapy.

In a particular embodiment, the present invention relates to the protein-based three-dimensional tissue scaffold of the present invention for use in therapy, wherein the protein-based three dimensional tissue scaffold is in the micronized form for administration by injection.

In a further embodiment, the present invention relates to the protein-based three-dimensional tissue scaffold of the present invention for use in tissue reconstruction.

In a particular embodiment, the present invention relates to the protein-based three-dimensional tissue scaffold of the present invention for use in tissue reconstruction, wherein the tissue reconstruction is a dermal replacement or substitute.

In a further particular embodiment, the present invention relates to the protein-based three-dimensional tissue scaffold of the present invention for use in tissue reconstruction, wherein the tissue reconstruction is used in the treatment of a wound.

In again a further particular embodiment, the present invention relates to the protein-based three dimensional tissue scaffold of the present invention for use in tissue reconstruction, wherein the wound is selected from a skin-loss wound, a chronic wound, a non-healing wound, a third degree burn, a fourth degree tissue loss, a traumatic skin-stripping wound and a surgical excision wound.

In again a further particular embodiment, the present invention relates to the protein-based three-dimensional tissue scaffold of the present invention for use in tissue reconstruction, wherein the protein-based three-dimensional tissue scaffold is positioned on the wound bed as acellular material.

In a further particular embodiment, the present invention relates to the protein-based three-dimensional tissue scaffold of the present invention for use in tissue reconstruction, wherein the tissue reconstruction comprises periosteum repair.

In a further embodiment, the present invention relates to a method of wound treatment, the method comprising positioning of the protein-based three-dimensional tissue scaffold of the present invention onto the wound bed.

In a particular embodiment, the present invention relates to a method of wound treatment, wherein the wound is selected from a skin-loss wound, a chronic wound, a non-healing wound, a third degree burn, a fourth-degree tissue loss, a traumatic skin-stripping wound and a surgical excision wound.

In a further particular embodiment, the present invention relates to a method of wound treatment, wherein the protein-based three-dimensional tissue scaffold is implanted on the wound bed as an acellular material.

### Definitions

In the disclosure of the present invention, the following definitions apply:

As understood herein, the term “aliphatic hydrocarbon chain” refers to a preferably saturated chemical moiety built of carbon and hydrogen atoms. As understood herein, a tertiary or a quaternary carbon atom refers to an sp3 carbon atom that is connected to one or no hydrogen atoms, respectively.

As understood herein, the term “at least one” is to be understood as one or more than one, unless otherwise indicated.

An emulsion as understood herein is a mixture of two or more immiscible phases; typically an oleic and an aqueous phase. Upon mixing, these liquids form a dispersion of droplets (the dispersed phase) in a continuous phase. The dispersed phase may also be referred to as internal phase.

As understood herein, the term “high-internal phase emulsion” or “HIPS” relates to an emulsion wherein the internal (or dispersed) phase constitutes at least 74% of the volume of the emulsion.

As understood herein, the term “high-internal phase emulsion regime” relates to conditions (for example temperature, pressure, presence of surfactants) that support the stability of a high-internal phase emulsion.

As understood herein, the term “hydrophilic-lipophilic balance (HLB)” describes the ratio between the size of the hydrophilic and lipophilic regions of a surfactant molecule. The empirically derived formula:

HLB=20×MW(Hydrophile)/MW

Was introduced for alkyl ethoxylates (Griffin W. C. J. Soc. Cosmet. Chem., 5 (1954), p. 249.) The HLB of more complex surfactant structures is found using a systematic approach based on chemical group assignments (Davies J. T., Rideal E. K. Interfacial Phenomena Academic Press, New York (1961), pp. 371-378):

HLB=7+Σ(Hydrophile group number)−Σ(lipophile group number)

wherein numbers for different groups are as previously reported (Davies, Rideal, 1961).

As understood herein, a “non-polypeptide polymer” polymer is a polymer comprising certain repeating units, wherein the repeating units are not connected by peptide bonds —C(O)NH—.

As understood herein, the term “polyethylene glycol” relates to a molecule or a moiety comprising more than one —OCH₂CH₂— repeat.

As understood herein, the term “scaffold” or “tissue scaffold” relates to a material that can promote wound healing by providing the environment for growth of cells and/or tissue.

### BRIEF DESCRIPTION OF FIGURES

FIG. 1: Stability of emulsions stabilised with 0.1% (v/v) surfactant at increasing oil fractions. Stability (TSI) was measured at 15 minutes after emulsification; lower TSI indicates higher stability. Emulsions were tested in triplicate.

FIG. 2: OCC for selected surfactants.

FIG. 3: Cross-sectional electron micrograph images and void diameter frequency charts of fibrin-based scaffolds produced by (A) foaming method and (B) emulsion templating. Foaming creates a high degree of porosity but offers no control over pore size or size distribution and therefore lacks reproducibility. Lack of pore size control is shown in the histogram with a wide distribution of void diameters recorded. Additionally, foaming can produce very dense regions of protein (shown in the higher magnification image). There are few interconnects or smaller voids. Emulsion templating offers more control over pore size, with a more uniform pore size distribution. Total void frequency is significantly higher in the templated scaffold owing to better interconnectivity and many more small voids forming the base-level of porosity.

FIG. 4: Cross-sectional scanning electron micrograph showing geometry of optimised emulsion-templated scaffold. A. Cumulative frequency from three sites analysed in the scaffold. B. Typical cross section of a freeze-dried scaffold.

FIG. 5: A. Cyto-compatibility of fibrin-based scaffolds based on 120 h culture in direct contact. Cell number was calculated from a standard curve, based on fluorescence of CCK-8 (1:10 dilution in DMEM) at 450 nm. Both (identical) emulsion-templated fibrin-based scaffolds (D3, D4) showed reduced cell count after 24 h in culture compared to the plate control. However, by 120 h cell numbers on the scaffolds had recovered close to plate control. Cell count increased on the ETPM 116% and 131% between 24 h and 120 h. Increase in cell count on the plate control in the same timeframe was 57%. B. SEM of h-tert MSC cultured on an emulsion-templated fibrin: PVA (1:1) scaffold at day 14, as described. The scaffold was fabricated according to the method describe for example 4, but with a lower concentration of fibrinogen (0.25% w/v clottable protein) but greater aqueous fraction of 20% (80% oleic phase).

FIG. 6: Macro-level pore structure, showing a range of pores from base level around 100 um up to 1000 um, resulting from coalescence of oil drops, as shown by digital imaging.

FIG. 7: Effect of fibrin concentration on scaffold architecture. A higher fibrinogen concentration results in the formation of a dense fibrous network of fibrin resembling a mat. Template fidelity is good at pore level, but there are few interconnects and diffusional gaps. Any additional materials such as co-polymers settle between the fibres and further reduce permeability (visible in 5000× image). At reduced fibrinogen concentration, template fidelity is preserved but the fibrin network is more diffusive and individual fibres are evident. Interconnectivity is greatly increased. Diffusional gaps are also far more prevalent.

FIG. 8: A: Number of blood vessels interacting with scaffold samples. Each timepoint represents the number of days after sample placement on the CAM (incubation day −4). Interacting blood vessels were defined those entering and exiting around sample circumference. Mean values and standard deviations reported; n variable owing to loss of samples and embryos; as reported in Table 1). Two-way ANOVA showed no significant differences between samples or time points. B. Cyanmethemoglobin (mg/ml) detected using Drabkin's Reagent at 540 nm (R 2=0.9995). Scaffolds were excised with forceps immediately prior to termination to minimise risk of contamination with blood from neighbouring severed blood vessels (mean and standard deviation). Two-way ANOVA revealed no statistically significant difference between samples.

FIG. 9: A: Tensile behaviour of fibrin-based scaffolds cross-linked with glutaraldehyde at various concentrations. Tensile testing of rectangular strips of freeze-dried scaffold samples showed an intermediate (0.8% v/v) glutaraldehyde concentration gave highest ultimate tensile strength (UTS) and Young's modulus compared to higher or lower concentrations. The large error bar arose from sample slippage in the vertical testing clamps; B: Effect of glutaraldehyde cross-linker concentration on tensile strength. Young's Modulus is consistently low (˜0.1 MPa) for all cross-linking concentrations except 0.8% glutaraldehyde (GTA). Ultimate tensile strength (UTS) increases with glutaraldehyde concentration up to 0.8% GTA but then reduces; C: Effect of cross-link concentration on surface area of fibrin-based scaffolds. Glutaraldehyde cross-linking causes moderate shrinkage at all concentrations; however, the reduction in area from as-cast dimensions is much less after the scaffolds have been freeze-dried.

FIG. 10: A: Effect of fibrin: PVA ratio on scaffold micro-architecture. Fibrin scaffolds without PVA are fragile and brittle but have a delicate basket-weave hierarchical structure (5000×, top row; 500×, bottom row). Interconnectivity is high, with high permeability from diffusional gaps. 1:1 fibrin: PVA has greatly reduced interconnectivity and diffusional gaps. The PVA glues together neighbouring fibrin strands, reducing the hierarchical structure. At and above fibrin content of 2:1 fibrin: PVA the bundling of fibrin fibres by PVA is still present, improving resilience and handle-ability, but the underlying fibrous architecture is still visible; B: Behaviour of bulk fibrin/PVA hydrogels under cyclic compressive loading. Bulk cross-linked hydrogels of 2:1 fibrin: PVA show an elastic response with good recovery between loading. This testing is qualitative and is shown only for a single sample to demonstrate typical behaviour.

FIG. 11: Proliferation of h-tert BM-MSC in scaffolds cross-linked by either EDC/NHS or glutaraldehyde as indicated in the legend. Scaffold were seeded with cells, and transferred after 24 hours to a fresh culture well. Culture medium was changed every 2 days. Cell number was determined by incubating cultures in CCK-8 reagent for 3 hours, and measuring the optical absorbance of a sample of supernatant. The assay was calibrated against a dilution series of measured cell numbers, after overnight seeding into tissue culture wells.

### DETAILED DESCRIPTION OF THE INVENTION

The invention is described in the following. It is to be understood that combinations of different features as described herein are also encompassed by the invention.

In a first embodiment, the present invention relates to a method for manufacturing a protein-based three-dimensional tissue scaffold. The method of the present invention comprises the steps of: (a) providing at least one protein capable of triggered polymerization; (b) forming a high-internal phase oil/water emulsion comprising the protein(s) of step (a) in aqueous phase, further comprising a surfactant which supports a high-internal phase emulsion regime; (c) triggering polymerization of the at least one protein comprised in the high-internal phase emulsion of step (b); and (d) washing out the oil phase.

In the step (a) of the method for manufacturing a protein-based three-dimensional tissue scaffold, at least one protein capable of triggered polymerization is provided. The protein capable of triggered polymerization is herein understood as any protein, preferably in a globular (i.e. non-membrane protein) form, that is preferably structurally substantially homogeneous (this form will be referred to as a non-polymerized protein), that upon an external factor may form a higher order structure. Said higher order structure is preferably a protein network, preferably formed around the oil droplets in the emulsion of the method of the present invention. Said higher order structure may be mediated for example by interactions between the non-polymerized proteins or their fragments. The higher order structure may also be mediated by formation of covalent bonds between the non-polymerized proteins. Protein polymerization may also be referred to as formation of elastic gel. Protein gelation may be induced to form a protein network around, and separate from, the oil droplets. Within the embodiments of the present invention, the step of protein gelation may include non-ionic, ionic or covalent crosslinking processes, or a combination thereof. In another example, the step of protein gelation may include enzymatic or non-enzymatic chemical reaction, ionic or non-ionic crosslinking processes, including thermally controlled molecular self-assembly, or a combination thereof.

In the method for manufacturing a three-dimensional protein based tissue scaffold of the present invention, protein polymerization, or protein gelation may be performed over a period of time under controlled conditions of temperature and humidity. Typically, the onset of physical gelation will be designed to occur within 5-10 minutes of casting, and completion of the gelation process may occur after up to 60 minutes after casting, for example 30 minutes after casting. Protein gelation may be preferably performed, at for example 37° C., in a humidified chamber, or tray covered to minimise evaporative loss from the scaffold surface.

The at least one protein capable of triggered polymerization, is selected for both its mechanical properties and its biocompatibility. Preferably, in the method for manufacturing a protein-based three-dimensional tissue scaffold of the present invention, the at least one protein capable of triggered polymerization of step (a) is selected from fibrinogen, collagen, laminin, elastin, cultured cell extracellular matrix extracts, and extracellular matrix preparations from natural tissue. More preferably, the at least one protein capable of triggered polymerization of step (a) is fibrinogen.

Fibrinogen as understood herein is as the coagulation factor I, a 340 kDa dimer comprising two identical sub-units consisting of three polypeptide chains designated Aα, Bβ, and γ (Mosesson-M W, J Thrm Haem 3:p1894-1904. https://doi.org/10.1111/j.1538-7836.2005.01365.x). There are two splice-variants of the γ-chain, a major variant, giving a homodimer with identical γ-chains of 411 aa, called fibrinogen type I. γ′ is a minor variant with an extended length in which the last 4 residues of the γ are replaced by an alternative 20 aa sequence. This variant occurs as a heterodimer giving fibrinogen type II, which forms a proportion around 15% of pooled fibrinogen (Chung-D W, 1984, Biochem. 23(18):4232-6. doi: Typically, fibrinogen is commercially purified from blood plasma by a selective precipitation method, such as cold precipitation, glycine/ethanol precipitation, β-alanine, ammonium sulphate precipitation (e.g. Keckwick-R A, 1955, Biochem. J. 60:671-678; Blombaeak-B, 1956 Arkiv. Kemi. 10: 415-443; Straughn-W, 1966, Thromb Diath Haematol. 16:198-206; Burnouf-Radosevich-M, 1990 Vox Sang 58:77-84). These methods are suitable for preparing fibrinogen from human and other species such as cattle, pigs, horses and dogs. Fibrinogen may also be produced through recombinant expression systems and is commercially available (e.g. Profibrix BV), although the economics of production may limit the use of such materials for bulk products such as wound scaffolds. Fibrinogen can be polymerized upon the addition of thrombin. Thrombin protease cleaves polypeptides referred to as fibrinopeptides A and B, generating activated fibrinogen, which self assembles via overlapping side-to-side non-covalent interactions with each other and thus forming a higher order fibre structures, in other words capable of polymerizing to form fibrin. Fibrin is the insoluble polymer of activated fibrinogen. This process is substantially identical to formation of blood clots in wound healing, as known to the skilled person. After injury, the coagulation and fibrinolytic cascades are important in providing rapid and appropriate haemostasis to minimise blood loss. If the vascular system is damaged, the coagulation cascade is activated, leading to the release and activation of thrombin (Factor IIa). Thrombin then acts to process soluble fibrinogen (Factor I) to fibrin, which then polymerizes. The fibrin fibrils recruit then other proteins to aid stability. Together, these actions form a clot, or thrombus, to seal the puncture and prevent further blood loss. Fibrinogen is one of the most abundant proteins in human plasma. It is synthesized by hepatocytes, at a rate of approximately 1.7-5 g/day. It then leaves the liver and circulates the body in blood plasma at a typical concentration of 2.5-3 mg/ml. When circulating cytokines such as IL-6 are provoked (i.e. in response to epidermal breach), pro-inflammatory agents are released which strongly upregulate the plasma concentration of fibrinogen. Elevated levels increase the potential for coagulation to occur.

The fibrinogen molecule comprises six chains with three identical pairs. These are denoted as Aα, Bβ and γ. The N-termini of all these chains face towards the centre of the fibrinogen molecule, the E domain. These chains intertwine from the central E domain, forming α-helical coiled-coil structures that are supported by disulphide bridges. This results in a generally linear molecule. However, while the Bβ and γ chains extend the length of fibrinogen, ending distally at one of two D regions, the Aa chains instead loop back towards the E domain. During the coagulation cascade, fibrinogen polymerises to fibrin. This occurs when thrombin cleaves the N-terminal sequences on the Aα and Bβ chains by proteolysis, releasing the surface-bound fibrinopeptides FpA and FpB, and in doing so exposing specific binding sites. The chain cleavage and release of FpB causes a drop in solubility that causes the molecules to aggregate laterally and increases the availability of α-chains to cross-linking by Factor XIII.

Preferably, in the method for manufacturing a protein-based three-dimensional tissue scaffold of the present invention the at least one protein capable of triggered polymerization of step (a) is fibrinogen. Herein, the step (c) is performed by addition of thrombin to the emulsion of step (b).

Preferably, the fibrinogen may be provided with additional calcium ions, preferably supplemented at a concentration of between 1 mM to 100 mM, in addition to calcium ions present in the preparations of fibrinogen as defined herein. It is known to the skilled person that the presence of calcium ions at certain concentration influences the clotting time of fibrinogen and together with the thrombin to fibrinogen ratio, gives rise to the production of fibrils with increased mass: length ratios. The resultant polymerized fibrin fibres, i.e. the resultant scaffold is therefore thicker and stronger. It is further noted that conversely, the presence of chloride ions leads to the production of thinner, less stiff, fibrin fibres. As known to the skilled person, this is due to the ions inhibiting aggregation of protofibrils during coagulation.

In the method for manufacturing a protein-based three-dimensional tissue scaffold of the present invention the at least one protein capable of triggered polymerization of step (a) can be collagen. Collagen is understood herein as the family of extracellular fibrous proteins that are characterized by their triple-stranded helical structure. Typically, three collagen polypeptide chains are wound around each other to form said triple-stranded helical structure. Collagens are highly conserved across mammalian species. However, the major structural collagens of connective tissue are part of a large family of collagens and collagen-related proteins with diverse functions. There are at least 28 collagen genes and different forms of collagen are deposited in different tissues, some are membrane associated, and present many different functions (Ricard-Blum, S., Cold Spring Harb. Perspect. Biol. 2011, 3(1):a004978). Therefore collagens which are typically extracted and used for tissue engineering represent examples of the collagen family proteins are fibre forming which may have utility for synthesisng scaffolds. Typically collagens of this use are type I, type II, type III, type IV type IX. Collagen is typically may be heterotypic, for example collagen from skin consists of type I and type III, from cartilage type II, IX and XI or II and III. Collagens are typically obtained and purified from acid extracted tissue such as skin, tendon ligament or cartilage, and is stably soluble in acidic solution. Acid collagens spontaneous assemble into fibre networks upon neutralization and warming. Therefore the controlled polymerization is performed by pH adjustment to neutrality under cold conditions such as 4° C., and warming when transferred to a casting mold.

In the method for manufacturing a protein-based three-dimensional tissue scaffold of the present invention the at least one protein capable of triggered polymerization of step (a) can be laminin. Laminin is herein understood as a protein or protein mixture from the laminin family. Laminins are heterotrimeric proteins formed from αβγ polypeptides, which form cross-shaped molecules which are key components of basement membranes, with distinct interactions with cell receptors. There are 11 human Laminin genes, 5α, 3β and 3γ. Functionally, laminins act as cell adhesion molecules which interact in the C terminal domains with integrin and at the N-terminal domains, with other extracellular matrix components (Aumailley-M, Cell Adh Migr, 2013, 7(1):48-55). The Engelbreth-Holm-Swarm sarcoma cell line was the source of the first laminin to be discovered and remains the source for commercial preparations such as Matrigel (BD Biosciences). Laminins are cold soluble and stable at 4° C., and gellate upon warming towards 37° C.

In the method for manufacturing a protein-based three-dimensional tissue scaffold of the present invention the at least one protein capable of triggered polymerization of step (a) can be elastin. Elastin is herein understood as a polypeptide product of the tropoelastin gene, of which there are multiple transcripts, with molecular weight in the range from 67 to 72 kDa. The elastic properties of elastin attributed to a repeating motif of Val-Pro-Gly-Xaa-Gly, has been used to synthesize synthetic elastin-like polymers, which would have utility for incorporating into scaffolds of the invention. One form of elastin, extracted under incubation in basic solution and elastase treatment, gellates upon acidification. Elastin may also be usefully combined with other ECM proteins such as fibrin or collagen, to confer enhanced elasticity and resilience of the resultant scaffold.

In the method for manufacturing a protein-based three-dimensional tissue scaffold of the present invention the at least one protein capable of triggered polymerization of step (a) can be cultured cell extracellular matrix extracts. The cultured cell extracellular matrix extracts are herein understood as reparations rich in laminin and type IV collagen, which can also contain other ECM components such as perlecan and entactin, and significant activities of growth factors such as TGF-β, VEGF and b-Fgf. Additionally, many adherent cells secrete ECM which accumulates over prolonged culture and may be extracted or solubilized, and used for fabrication as a major or additional component of synthetic matrices and scaffolds. Typically, extracts will be dissolved in cold conditions as a viscous solution and stored frozen, and will self-assemble and gellate upon warming to room temperature or above.

In the method for manufacturing a protein-based three-dimensional tissue scaffold of the present invention the at least one protein capable of triggered polymerization of step (a) can be extracellular matrix preparations from natural tissue. The extracellular matrix preparations from natural tissue are herein understood as any method in which tissue is treated to extract the extracellular matrix material, with the aim of achieving a preparation with greater complexity than a purified fraction, to reconstitute an organ-specific induction of tissue regeneration. Extraction methods may include a detergent or solvent extraction, enzymatic digestion, or acidic or basic lysis, osmotic shock, salt extraction, It is understood that such extraction processes involve decellularisation and removal of antigens, in order to render the extract safe for allogenic use (Chen & Liu, 2016 Prog Polym Sci. 53: 86-168. doi:10.016/j.progpolymsci.2015.02.004).

In the method for manufacturing a protein-based three-dimensional tissue scaffold of the present invention the amount of the at least one protein capable of triggered polymerization in the final aqueous phase of the oil-in-water emulsion mixture is preferably in the range of 0.01 to 5% w/v, preferably 0.05 to 4% w/v, more preferably to 3% w/v. Preferred range may depend on the at least one protein capable of triggered polymerization used in the method of the present invention. For example, a preferred range may be 1 mg/ml (0.1% w/v) to 5 mg/ml (0.5% w/v) for collagen type I, and 10 mg/ml (1% w/v) to 50 mg/ml (5% w/v) for fibrinogen.

It is noted that scaffold materials remain in the wound upon would regeneration, and may often separate and elicit an inflammatory response. This may in particular happen for scaffolds created using certain polymeric materials, such as poly-L-lactic acid (PLLA), poly-glycolic acid (PGA), polycaprolactone (PCL) and copolymer of lactic and glycolic acid (PLGA), which degrade into acidic by-products. Appropriate material selection, as well as other features of the scaffold including degree of cross-linking and scaffold dimensions (for example wall thickness) help ensure scaffold materials do not persist in the healed wound. Scaffolds for tissue regeneration should instead degrade steadily during healing and leave nothing remaining. This allows space for generation of new extracellular matrix products.

Scaffolds of the present invention are preferably capable of maintaining a hydrated wound environment, replacing the hydrating properties of intact dermis. Herein, a hydrated environment is understood as an environment in which aqueous medium such as tissue fluid, is contained within the porous structure of the scaffold, as distinct from the bound water which is tightly associated with the protein structure of the lyophilised scaffold, constituting less than 10% and around 5% of the dry weight. This may be particularly beneficial in reducing formation of scars. As understood herein, it is also preferred for scaffolds to have the capacity to absorb wound debris such as exudate. Absorption of exudate is an additional advantage of the porous sponge-like structure most scaffolds adopt. Exudate is herein understood as any fluid that is released from the circulatory system into lesions, wounds or areas of inflammation. When an injury occurs, leaving skin exposed, it leaks out of the blood vessels into nearby tissues. Typically, exudate is composed of serum, fibrin and leukocytes. It is understood that a scaffold of the invention in clinical use will preferably be covered by an occlusive or semi-occlusive secondary dressing, such as a silicone mesh.

Certain limitations as to for example pH and/or temperature maintained during the method for the manufacture of a three-dimensional protein-based tissue scaffold may have to be applied in order to retain the integrity of the material used and ensure its biocompatibility. At elevated temperatures, proteins are likely to denature, which may alter their structure such that their desired function is impaired or lost. It has been found that in scaffolds made of gelatin, a denatured form of collagen, cell proliferation was lower than in comparable scaffolds of a less processed form of collagen. Preferably, during the method for the manufacture of the three-dimensional protein-based scaffold, the temperature as well as pH are maintained in physiological ranges. Preferably, the temperature is maintained in the range 4° C. to 37° C., and/or the pH is maintained in the range from 6 to 8.

The method for manufacturing a protein-based three-dimensional tissue scaffold further comprises the step (b) of forming a high-internal phase oil/water emulsion comprising the protein(s) of step (a) in aqueous phase. The said high internal phase oil-water emulsion further comprises a surfactant which supports a high-internal phase emulsion regime.

An emulsion as understood herein is a mixture of two or more immiscible phases; typically an oleic and an aqueous phase. Upon mixing, these liquids form a dispersion of droplets (the dispersed phase) in a continuous phase. The dispersed phase may also be referred to as internal phase. Emulsions are thermodynamically unstable systems and inevitably tend towards phase separation. The stability of the emulsion is determined by the rate at which it decays; i.e. the droplets change size and distribution by processes such as creaming and sedimentation, coalescence and flocculation, as known to the skilled person. Gravitational separation occurs when the dispersed particles migrate upwards (“creaming”) or downwards (“sedimentation”) depending on their relative density to the continuous phase. Droplets may also clump together to form aggregates. If the droplets retain their original integrity, this process is known as “flocculation”. If the droplets merge together and grow, the process is “coalescence”. Ostwald ripening is another process of droplet growth, whereby smaller droplets feed larger ones by mass transport through the continuous phase. Emulsions can be destabilised by one or many of these processes.

The ability of emulsions to form droplets of readily controllable size gives many applications in cell and tissue engineering, including templated scaffolds, where the continuous phase creates a porous sponge-like structure after removal of the dispersed phase.

As known to the skilled person, emulsions with smaller droplet diameters in the dispersed phase are more stable. An amphiphilic molecule, such as a surfactant, may be added to promote stability of the emulsion. Surfactants have both lipophilic and hydrophilic regions, allowing organized assembly at the interface between the oil and aqueous phases. The barrier they create slows instability. The ratio between the size of the hydrophilic and lipophilic regions of a surfactant molecule is expressed by as HLB (hydrophilic-lipophilic balance). Preferably, the emulsion as used in the method of the present invention is an oil-in-water emulsion. An oil-in-water (o/w) emulsion has oleic droplets dispersed in an aqueous continuous phase. Preferably, in the method of the present invention, any oil phase that has a freezing point higher than −50° C., more preferably higher than −30° C., can be used. Preferably, the emulsion as used in the method of the present invention comprises n-decane as an oleic phase/dispersed phase/internal phase. Therefore, within the scope of the present invention it is understood that oil phase as understood herein can be freeze dried.

The emulsion as used in the method for producing a three-dimensional protein-based scaffold of the present invention can be a mesoemulsion. As understood to the skilled person, a mesoemulsion as used in the method of the present invention comprises droplets of a mean diameter of between 1 and 50 microns. As disclosed herein, droplet size understood as droplet diameter was determined (computationally, according to Mie theory) using Turbiscan results, using a combination of backscattering and transmission data, and the refractive indices of the continuous and dispersed phases. Alternatively, the emulsion as used in the method for producing a three-dimensional protein-based scaffold of the present invention can be a macroemulsion. As known to the skilled person, the macroemulsion comprises droplets of an average diameter of at least 50 microns. Therefore, the emulsion as used in the method for producing a three-dimensional protein-based scaffold of the present invention preferably comprise the droplets of an average diameter of at least 1 micron, more preferably of at least 50 micron. Further preferably, the emulsion as used in the method for producing a three-dimensional protein-based scaffold of the present invention preferably comprises the droplets of an average diameter of between 20 to 250 μm, more preferably of between 70 to 150 μm. In practice, the emulsion characteristics for templating are preferably determined by the upper limit of dispersed phase droplet diameter. The lower limit of droplet diameter is preferably determined by the shear rate in the device or geometry used to create the emulsion, whereas the upper limit is preferably determined by the circulation of the mixture through the limiting shear rate zone, and the stability of the formed droplets. The mean diameter can be estimated from light microscopic examination and image analysis of captured images. The mean diameter can be calculated more accurately by the weighted mean of droplet diameters of a variable number measured in a field of view, for several fields, according to the formula:

Dₘₑₐₙ=(F1ₘₑₐₙ×F1ₙ)+(F2ₘₑₐₙ×F2ₙ)/(F1ₙ+F2ₙ)

In the method for manufacturing a three-dimensional protein-based scaffold of the present invention, a surfactant which supports the high-internal phase emulsion regime is to be used. Any surfactant that can be used to support the high-internal phase emulsion regime can be used in the method of the present invention. Preferably, in the method for manufacturing a protein-based three-dimensional tissue scaffold, the surfactant which supports the high-internal phase emulsion regime of step (b) comprises: at least one optionally substituted polyethylene glycol chain comprising 5 to 16 ether bonds, and at least one aliphatic hydrocarbon chain of at least 8 carbon atoms, wherein at least one tertiary or quaternary carbon atom is present in the at least one aliphatic hydrocarbon chain. The balance between the hydrophilic and hydrophobic part of the surfactant is not sufficient to determine whether a surfactant is able to support the high-internal phase emulsion regime, although the surfactant HLB should preferably fall within certain broad limits, for the particular oleic phase (for example 12.5-16 for n-decane).

A water-in-oil emulsion (w/o) has oil droplets dispersed in an aqueous continuous phase. An oil-in-water (o/w) emulsion has aqueous droplets dispersed in an oleic continuous phase. The propensity for formation of a w/o or o/w emulsion is determined by the balance of hydrophobicity and hydrophilicity of the solutions in the mixture. This may be determined using the HLB scale. Oleic phase reagents are assigned an HLB number, determined experimentally, that allows for selection of an appropriate surfactant to cause emulsification with the aqueous phase. This is not a precise method; factors such as temperature, and salt content of the aqueous phase, alter the required HLB. HLB as understood herein is a semi-quantitative measure of the relative molecular weights of the hydrophilic and lipophilic regions of a surfactant molecule assigned on a scale from 0.5 (extremely lipophilic) to 19.5 (extremely hydrophilic).

A water-in-oil emulsion (w/o) has oil droplets dispersed in an aqueous continuous phase. An oil-in-water emulsion has aqueous droplets dispersed in an oleic continuous phase. The propensity for formation of a w/o or o/w emulsion is determined by the balance of hydrophobicity and hydrophilicity of the solutions in the mixture. This may be simply determined using the HLB scale. Oleic phase reagents are assigned an HLB number, determined experimentally, that allows for selection of an appropriate surfactant to cause emulsification with the aqueous phase. This is not a precise method; factors such as temperature, and salt content of the aqueous phase, alter the required HLB. Furthermore, HLB only describes the behaviour of surfactants in aqueous solutions and does not consider the environment (including salinity or temperature of the system) or other surfactant characteristics such as structure

In an emulsion as defined herein, droplets of the dispersed phase are typically spherical. This minimises the surface area of contact between the continuous and internal phase and is most thermodynamically favourable. Assuming uniform droplet diameter of perfect spheres, the maximum volume fraction of the internal phase 74%. Emulsions with an internal phase exceeding 74% total volume are referred to as high internal phase emulsions (HIPEs). The spherical droplets become deformed into polyhedra and the continuous phase is compressed into thin films at the interface. The rheological properties change dramatically in HIPEs. HIPEs exhibit high storage modulus and yield stress under low shear, so droplet deformation is insufficient to result in bulk flow. Under high shear when the yield stress is exceeded, the emulsion changes from displaying elastic to viscous behaviour). These properties arise from the combined effect of some independent variables (internal phase volume fraction) and some dependent variables (interfacial tension, droplet size and range of droplet size).

Stability of emulsions is very important for emulsion templating. An emulsion that undergoes creaming or sedimentation will give raise to a heterogeneously porous scaffold, with certain parts having much lower porosity than the other parts. Instability arising from coalescence or flocculation will give rise to irregular pore size and non-homogeneous pore distribution, or loss (i.e. reduction) of the desired volumetric porosity. This would produce varied wall thicknesses, with some too thin and liable to rupture, and others too thick to allow diffusion and potentially thick enough to provoke an adverse immune response.

In again a further particular embodiment, the present invention relates to a method for manufacturing a protein-based three-dimensional tissue scaffold, wherein the surfactant which supports the high-internal phase emulsion regime of step (b) exhibits oil-carrying capacity (OCC) in the range from 75% to 95% and preferably hydrophilic-lipophilic balance (HLB) in the range from 12 to 16.

Preferably, in the method for manufacturing a protein-based three dimensional tissue scaffold, the oil-in-water emulsion of the present invention comprises a surfactant which supports the high-internal phase emulsion at the concentration of between 0.1% to 10% v/v. Preferably, the oil-in-water emulsion of the present invention comprises a surfactant which support the high-internal phase emulsion at the concentration of between 0.05% to 1% volume %, more preferably 0.1% to 0.5% volume % of the total volume of the oil in the oil-in-water emulsion. Where a blend of surfactants is employed, preferably the total amount of surfactant may be in the range of 0.05-2% mass of the oil phase. Within the scope of the present invention, the surfactant which supports the high-internal phase emulsion may optionally be employed in combination with a stabilising agent. A suitable stabilising agent may be polyvinylpyrrolidone. The stabilising agent may be employed in an amount of 0.1 to 10% w/v of the aqueous phase, for example, 0.5 to 5% w/v of the oil-in-water emulsion.

Herein, a supplementary surfactant screening tool, “oil carrying capacity”, is proposed. Using a test emulsion comprising decane, aqueous buffer and common industrial surfactants, we assessed short-term dynamic emulsion stability at increasing oil fractions using static multiple light scattering. This system of determining the oil fraction at which optimum emulsion stability occurs, the ‘oil carrying capacity’ of each surfactant, is intended as a tool for surfactant selection in a system where the desired oil fraction is known. Equally, it may be used to optimise oil fraction ‘carried’ in the emulsion when a particular surfactant is required.

In the examples discussed herein, surfactants from HLB 9.8 to 17.6 were included. Emulsion stability was used to rank surfactants for their ability to generate high internal phase oil-in-water emulsions in the test system.

Preferably, in the method for manufacturing a protein-based three-dimensional tissue scaffold of the present invention, the surfactant which supports the high-internal phase emulsion regime of step (b) is selected from tergitol-NP10, Tergitol-TMN-6; Tergitol-TMN-10, triton-X100-R, Triton X104R, Triton X114R, Triton-CG110, Triton X165, and Ecosurf-EH-9. However, this list is not to be construed as limiting and it is to be understood that other surfactants with suitable properties, for example with a suitable OCC value, for example polyglycerydyl 4-laurate, can be used within the scope of the present invention. Preferably, the surfactants useful in the method for manufacturing a protein-based three-dimensional tissue scaffold of the present invention are non-ionic detergents. However, in certain embodiments of the present invention, should a mixture of surfactants be used, not more than 10% of the surfactant may be an ionic surfactant(s).

It is understood herein that the names of surfactants as referred to herein are commercial names and are valid as of the date of filing of the present application, and may be changed with time. Therefore, in the Tables 1 and 2 below, chemical structures of the surfactants discussed herein are presented for clarity.

TABLE 1
Structures and HLB parameters of the surfactants as described herein.

| Surfactant | | HLB | Linear formula | Skeletal formula |
|---|---|---|---|---|
| Tergitol | 15-S-7 15-S-9 15-S-15 | 12.1 13.3 15.4 | C₁₁₋₁₅H₂₃₋₃₁O(CH₂CCH₂O)₇H C11-15 pareth-7 C₁₁₋₁₅H₂₃₋₃₁O(CH₂CCH₂O)₉H C11-15 pareth-9 C₁₁₋₁₅H₂₃₋₃₁O(CH₂CCH₂O)₁₅H | 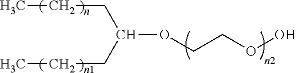 |
| | | | C11-15 pareth-15 | |
| Tween | 20 40 80 | 16.7 15.6 15 | C₅₈H₁₁₄O₂₆ Polyethylene glycol sorbitan monolaurate C₂₂H₄₂O₆•(C₂H₄O)ₙ, n = ~20 Polyoxyethylene sorbitan monopalmitate C₆₄H₁₂₄O₂₆ Polyethylene glycol sorbitan oleate | 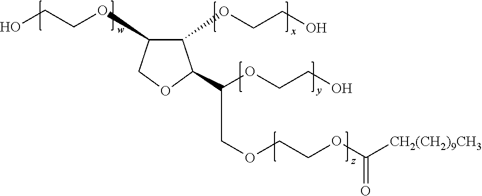 |
| Brij | O10 O20 58 | 12.4 15 15.7 | C₁₈H₃₅(OCH₂CH₂)ₙOH Polyoxyethylene (10) oleyl ether C₁₈H₃₅(OCH₂CH₂)ₙOH Polyoxyethylene (20) oleyl ether C₁₆H₃₃(OCH₂CH₂)ₙOH Polyoxyethylene (20) cetyl ether | 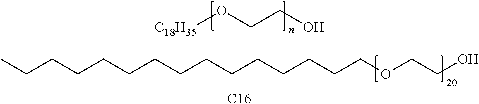 |
| Triton | X-45 X-100 X-102 X-114 X-165 X-405 | 9.8 13.4 14.4 12.3 15.5 17.6 | (C₂H₄O)ₙ C₁₄H₂₂O, n = ~5 Polyethylene glycol 4-tert- octylphenyl ether t-Oct-C₆H₄—(OCH₂CH₂)ₙOH, n = 9-10 Polyethylene glycol tert- octylphenyl ether | 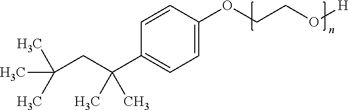 |
| | | | (C₂H₄O)ₙ C₁₄H₂₂O, n = 12 | |
| | | | Octylphenol ethoxylate | |
| | | | (C₂H₄O)ₙ C₁₄H₂₂O, n = 7-8 | |
| | | | Polyethylene glycol tert- | |
| | | | octylphenyl ether | |
| | | | (C₂H₄O)ₙ C₁₄H₂₂O, n = 16 | |
| | | | Octylphenol ethoxylate | |
| | | | (C₂H₄O)ₙ C₁₄H₂₂O, n = 35 | |
| | | | Polyoxyethylene (40) | |
| | | | isooctylphenyl ether | |

TABLE 2
Structures and HLB parameters of the surfactants as described herein
(selection of surfactants also referred to herein as group B).

| Surfactant | | HLB | Linear formula | Structure |
|---|---|---|---|---|
| Tergitol | TMN-6 TMN-10 NP-10* | 13.1 14.4 13.2 | C₁₁₋₁₅H₂₃₋₃₁O(CH₂CCH₂O)₇H Polyethylene glycol trimethylnonyl ether C₁₁₋₁₅H₂₃₋₃₁O(CH₂CCH₂O)₉H Polyethylene glycol trimethylnonyl ether C₉H₁₉•C₆H₆•(OCH₂CH₂)ₙOH Nonylphenol ethoxylate | 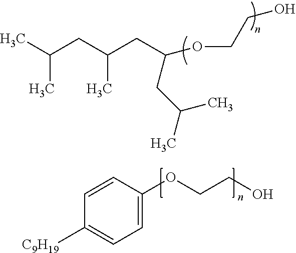 |
| Triton | X-100 reduced X-114 reduced N-101 reduced | 9.8 13.4 14.4 | (C₂H₄O)ₙ C₁₄H₂₂O, n = ~5 Polyethylene glycol 4-tert-octylphenyl ether t-Oct-C₆H₄—(OCH₂CH₂)ₙOH, n = 9-10 Polyethylene glycol tert-octylphenyl ether | 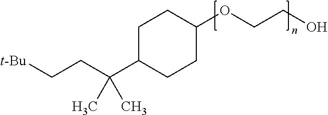 |
| | | | (C₂H₄O)ₙ C₁₄H₂₂O, n = 12 | |
| | | | Octylphenol ethoxylate | |
| Ecosurf | EH-9 | 12.5 | Ethylene oxide-propylene oxide copolymer mono(2-ethylhexyl) ether | 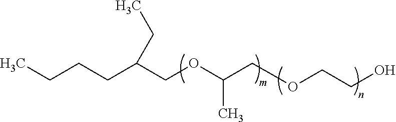 |
| Triton | CG110 | | Alkylpolyglucoside** | |

*C₉H₁₉ is preferably meant here as a branched saturated hydrocarbon chain of 9 carbon atoms.
**According to the general description of the manufacturer (Dow) it is a mixture of D-glucopyranose, oligomeric, decyl octyl glycoside.

The method for manufacturing a protein-based three-dimensional tissue scaffold of the present invention further comprises the step (c) of triggering polymerization of the at least one protein comprised in the high-internal phase emulsion of step (b). As exemplified herein, this can be achieved upon addition of thrombin to the high-internal phase emulsion comprising fibrinogen. This way, fibrinogen is processed to fibrin which polymerizes. The rate of polymerization is determined by the conditions within the aqueous phase, such as concentration of calcium ions, concentration or specific activity of thrombin and ation of thrombin to fibrinogen, as well as the temperature of the system. However, this example is not meant to be construed as limiting in any way, and any other way of triggering polymerization of the at least one protein, as it can be known to the skilled person, can be used in the method of the present invention.

The method for manufacturing a protein-based three-dimensional tissue scaffold of the present invention further comprises the step (d) of washing out the oil phase. emulsion oil droplets may be removed from the scaffold after formation. Preferably, the oil droplets may be removed by elution from the scaffold. Any suitable solvent may be used for this step. For example, an alcohol solvent may be used. The alcohol may be an aliphatic alcohol. In one embodiment, the alcohol is a C1-C12 alcohol, preferably a C1-C6 alcohol, even more preferably a C1-C4 alcohol. Examples of suitable alcohols include methanol, ethanol, propane-1-01, propane-2-ol, butane-1-ol, butane-2-ol, tert-butanol and mixtures thereof. The elution is preferably performed by washing the scaffold with an excess volume of the alcohol or alcohol solution. The washing step may be aided by gentle agitation, such as achieved by rotary orbital motion. Alternatively, residual oil phase can be removed by freeze drying, in particular when the oil phase is as defined herein, i.e. has a freezing point higher than −50° C., more preferably higher than −30° C., even more preferably is n-decane.

Preferably, following the removal of oil droplets in step (d), the so obtained three-dimensional protein-based scaffold of the present invention is incubated with an excipient solution. The excipient may be a water-soluble organic hydrophilic excipient. Examples of suitable excipients include polyols such as sugars (e.g. mannitol, sorbitol, dextrose, sucrose), polymers such as polyvinyl alcohol (PVA), polyethylene glycol (PEG) or polyoxyethylene-polyoxypropylene glycol block co-polymer (e.g. pluronic P68), polyvinylpyrrolidone (PVP) (e.g. Kollidon™), or polyethylene glycol-polyvinyl alcohol co polymers (e.g. Kollicoat™). A mixture of excipients may be used. The concentration of the excipient in solution, and then may be up to 10 volume %, for example from 1 to 5 volume %. According to the present invention, the scaffold may be incubated for any suitable amount of time, for example for 5 to 10 minutes. The excipient may reduce bulk shrinkage of the scaffold during drying and may also preserve the nano-structure of the scaffold during a freeze-drying process. The concentration of excipient used is in the range 0.5 to 2 M aqueous for sugars, or in the range of 0.5 to 5 w/v aqueous for polymeric excipients. Following incubation, excess excipient solution is preferably removed from the scaffold, for example by draining, while maintaining saturation of the scaffold with the excipient solution. The excipient itself coats the scaffold structure, and can be removed after freeze drying, for example can be removed by washing. Removal of the excipient is desirable before use of the scaffold in, for example, treatment of wounds.

The so obtained (or so obtainable) protein-based three-dimensional tissue scaffold can be further used. Preferably, the so obtained (or the so obtainable) is processed further, as described herein, before it is used.

Preferably, the method for manufacturing a protein-based three-dimensional tissue scaffold of the present invention further comprises a step of crosslinking the protein-based three-dimensional scaffold obtained in step (d). As discussed herein, the additional crosslinking step may join (e.g. covalently) protein molecules together to create an insoluble matrix. It understood in the art that cross-linking between proteins may allow control of the bulk proteolytic degradation rate of a protein scaffold. Cross linking can increase the physical strength and chemical and biochemical stability of the protein scaffold. It is believed that this may allow control of the degradation rate and profile of the scaffold. Crosslinking may also increase the mechanical properties of the scaffold. For this purpose, any suitable protein cross-linking agent can be used. The examples include, but are not limited to, chemical cross-linking agents such as genipin, glutaraldehyde or carbodiimides such as EDC in NHS buffer can be used. Alternatively, photo crosslinking with the aid of a ruthenium catalyst can be performed. In certain embodiments, a fibrin-based three dimensional tissue scaffold can be cross-linked as described herein. It should be noted that in vivo, cross-linking of newly-formed soluble fibrin fibrils occurs by activated coagulation factor XIII, which stabilises the fibrils to form a strong, elastic insoluble clot. The same effect may be achieved in artificial fibrin constructs by the reagents and treatments as described herein. Cross-linking of the three-dimensional protein-based scaffold as described herein can alternatively also be performed before the step (d) of the method for manufacturing the three-dimensional protein-based scaffold of the present invention. This may be achieved by introducing a cross-linking agent into the initial protein coagulation mixture, or prior to elution of the oil phase.

Further preferably, the method for manufacturing a protein-based three-dimensional tissue scaffold of the present invention further comprises a step of freeze-drying the protein-based three-dimensional scaffold. Freeze-drying (or lyophilisation) is a dehydration process that works by freezing the scaffold which is saturated in water or excipient solution, and then reducing the surrounding pressure. This allows the removal of solvent (water) and remaining volatile oil from the scaffold. Thus, freeze-drying advantageously improves the storage and shelf life of the scaffold. Controlling the freeze-drying parameters allows for preservation of the scaffold nanostructure that has been formed by emulsion-templating. Preferably, the freeze-drying is performed at between −20 to −40° C., and at a pressure below the corresponding vapour pressure at the selected drying temperature, such as at <200 mTorr. For example, freeze-drying can be performed at −40° C. at <100 mTorr for 10 hr, and then the temperature is increased to −30° C., −20, 0° C. and 20° C. in a series of steps, with at least 1 h hold between each step.

Further preferably, the method for manufacturing a protein-based three-dimensional tissue scaffold of the present invention may further comprise additional treatment steps. Treating the external and internal surface of a bulk biomaterial affects the way it interacts with cells. Surface treatment can be used to enhance performance as tissue scaffold, or to broaden the selection of appropriate materials by providing a biocompatible surface to a substrate. Substrate is herein understood as environment where culture cells may live, grow and/or propagate and organize into morphological structures such as blood capillaries. Chemically or bio-chemically modifying the material surface has been shown to influence cellular adhesion, allowing control in the way cells interact with the implant. Biomaterial surfaces may also be biologically treated, for example with the immobilisation of growth factors, to promote or otherwise influence cell adhesion, proliferation and behaviour. Cells respond in a very sensitive manner to biomaterial surfaces. Topography of said surface can govern the mode of adhesion, the extent and direction of migration, and even differentiation. The use of non-protein polymers in addition to protein(s) can influence the organization of the nanoscale structure, to influence internal surface area, topology, and surface activity.

Further preferably, the method for manufacturing a protein-based three-dimensional tissue scaffold of the present invention may further comprise the step of addition of protease inhibitors to the scaffold. As known to the skilled person, one problem with the use of polypeptides, in particular fibrin for implantable biomaterials is its rapid protease-mediated degradation in vivo, i.e. upon therapeutic application. Protease inhibitors like aprotinin or tranexamic acid can delay proteolytic degradation, prolonging the persistence of implanted scaffolds, in particular scaffolds comprising fibrin constructs, as required to support the repairing tissue.

In a further embodiment, the present invention relates to a three-dimensional scaffold obtainable according to the methods of the present invention, as described herein. Preferably the protein-based three-dimensional scaffold obtainable according to the method for manufacturing a polymerized protein-based three-dimensional tissue scaffold of the present invention has a scaffold porosity of at least 74%. The porosity of the scaffold obtained preferably in lyophilised form is assessed by SEM measurement of large pore, interconnecting pores and base-level porosity.

Herein, the “porosity” is understood as ratio of the volume of the pores to volume of the entire scaffold including the pores volume and is commonly expressed in percent. The porosity as understood herein may also be referred to as “volumetric porosity”.

Porous scaffolds are widely used in in the treatment of injured or diseased tissue. They can be used as a physical plug to fill an extensive cavity and prevent at least in part contraction and/or scarring. They are further used to provide a suitable structure to allow for cell infiltration and encourage tissue regeneration.

Porosity can be introduced in a scaffold through emulsion templating. Herein, the dispersed phase acts as a set of liquid phase porogens. Porogens are herein understood as particles that can be used to make pores in a certain structure, herein in a scaffold. Thus, the emulsion creates a ‘template’ for the scaffold. The size and shape of the porogen determines the size and shape of the resultant pore. The density and packing arrangement of the porogens determines the number, spacing and interconnectivity of the pores. The versatility of emulsion templating arises from the ability of emulsions to form readily controllable droplets. Either hydrophilic or hydrophobic polymers may be used depending on the emulsion type used. Hydrophilic polymers dissolve in the aqueous phase to form a scaffold matrix around oil droplets in an o/w emulsion. In the methods of the present invention, at least one protein is used as a hydrophilic polymer.

As known to the skilled person, beyond emulsion templating porosity can also be introduced to a scaffold by particulate leaching, gas bubbling, phase separation, melt moulding, and foaming.

In a porous templated scaffold, the volume fraction of the dispersed phase of the emulsion determines scaffold porosity and also influences wall thickness by closeness of adjacent droplets. Wall thickness is herein understood as thickness of the material layer separating adjacent pores. Wall thickness as defined herein can be directly determined by SEM. Droplet diameter influences and/or determines the pore diameter. As discussed herein, the average diameter of dispersed droplets in an emulsion is typically at least 1 μm, more preferably at least 50 μm. The average diameter is herein understood as the mean diameter, as defined herein. The droplet size distribution is often broad. However, in a macroemulsion system, the presence of mesoemulsion droplets pack in the intertices without undue adverse effect on the scaffold structures. Additionally, additional processing, such as fractionation, may be employed to separate and remove droplets with diameters outside the desired range. Emulsion droplet size can be controlled and varied by altering the interfacial tension between the oleic and aqueous phases. For example, increasing surfactant concentration reduces interfacial tension between the dispersed and continuous phase leads to a reduction in the diameter of the droplets of the dispersed phase.

Preferably, the scaffolds produced according to the methods of the present invention exhibit a narrow pore size distribution. However, as different sizes for pore and interconnect diameter are desired as known to the skilled person, it may be desirable to provide a scaffold wherein the distributions of pore diameters is a bimodal distribution, in other words includes two maxima, around two pore diameter values. This way, both pores optimised for angiogenesis and vascularization, preferably of an average diameter larger than 100 μm, as well as pores of an average diameter not larger than 100 μm optimized for cell migration and settlement would be present. The spatial distribution of these differently sized pores may have to be optimised to support formation of homogenous tissue. Additionally, the mesoscale porous structure is formed from a fibre-mesh structure, which sets a wall porosity in the mesoscale range, with pore diameters below 10 μm.

The number and distribution of pores is directly linked to the cell-loading capability of a scaffold (understood as number of cells that scaffold can be populated with per unit of volume), and also the spacing between these cells. The density of cells in a scaffold affects the rates of cell proliferation and deposition of new ECM. These two factors play a major role in the properties of the resultant tissue. In determining a therapeutic effect of a cell-seeded scaffold, the optimal density of therapeutic cells will need to be determined, with an upper limit determined by the internal surface area of the scaffold structure.

Porous materials are highly desirable as scaffolds. As defined herein pores as interconnected cavities provide space for vascularisation and angiogenesis, and aid the supply of nutrients and chemical stimuli and removal of waste from the regenerating tissue. A highly porous material will allow for increased and more uniform infiltration of cells. As known to the skilled person, vascularisation is key to the success of a tissue engineered construct—insufficient vascularisation results in impaired exchange of nutrients and waste and impedes immune response. As a result, infection, localised necrosis or even loosening and rejection of the implant may occur. Vascularisation of acellular scaffolds may be encouraged by modification of the structural or physicochemical properties of the scaffold or by release of growth actors

The pore structure as defined herein is important to properly accommodate cells, enabling them to migrate through, adhere to and interact with the scaffold. The degree of porosity, and the size of the pores, can affect the behaviour of cells in a scaffold. Pore size is therefore an important criterion when developing a scaffold for use with any cell type. In particular, pore size is an important criterion when developing a scaffold for use with stem cells. The pore sizes larger than 150 μm tend to give rise to the formation of a low-density network of large blood vessels which are capable of penetrating deep into the scaffold. Pore sizes less than 150 μm give rise to high-density networks of small blood vessels, but penetration depth is reduced. As such, large pore sizes are desirable for large three-dimensional scaffolds, whereas smaller pore sizes are more suited to smaller, thinner scaffolds such as those required for skin substitutes.

Preferably, scaffolds of the present invention have average pore sizes of between 50 μm and 400 μm, more preferably of between 100 μm and 300 μm. Scaffolds with the average pore sizes of between 100 μm and 250 μm are particularly suitable as scaffolds for skin tissue.

Further to pore size, the size of the interconnections is almost important is promoting vascularisation of the scaffold. Large interconnections (preferably larger than 30 μm, more preferably larger than 50 μm) show enhanced vascularisation in comparison to scaffolds with smaller interconnections. This is likely a direct result of growing blood vessels being presented with a more open path into the implanted scaffold. Herein, interconnections are understood as gaps between adjacent large pores, also understood as smaller pores that interconnect adjacent large pores. Their appearance is controlled by the formed density of the protein fibre mesh.

The interconnection size in certain cases may be a more important parameter than pore size. Optimised interconnect diameter promotes vascularisation by promoting proliferation, adhesion and migration of endothelial cells through scaffolds. Scaffolds with larger interconnections have been shown to exhibit improved vascularisation over those with smaller diameter interconnections. In the scaffolds of the present invention, interconnections are of a size of between 20 μm and 400 μm, more preferably 30 μm and 200 μm, even more preferably 50 μm and 150 μm. It is observed that improved vascularization is observed with increased interconnection size, however beyond 400 μm no further significant difference is observed.

Further to the interconnecting pore size, the fibre mesh porosity equates the wall porosity sets the base-scale of the scaffold structure in the nano-scale, since each fibril diameter is in the range 100-150 nm. This length scale provides a suitable scale for cellular interactions, and influencing cell differentiation, spreading and cell migration.

High volumetric porosity is achieved by maximising the surface area-to-volume ratio of the dispersed phase. In the method of the present invention therefore very little protein material is therefore required to create a scaffold. The scaffold must be sufficiently tough to be readily handleable, but also must provide a suitable physical environment for cell ingress and formation of new tissue in vivo.

Preferably, the three-dimensional protein-based scaffolds obtainable according to the methods of the present invention exhibits a surface area of 0.04-0.06 m 2/ml in the lyophilised state. The surface area as defined herein is the internal surface of the scaffold created by the higher order pore structure and base level fibre mesh structure. This represents the maximal internal surface area accessible for cell adhesion. This can be estimated through geometric calculation on the basis of emulsion oil droplet average diameter or average pore diameter, and preferably determined by a BET method. BET method relates to the measurement of surface area according to Brunauer-Emmet-Teller theory, herein preferably measured by a reversed phase absorption BET analysis, using inverse gas chromatography SEA (Surface measurement Systems Ltd, Alperton, UK).

Further preferably, the three-dimensional protein-based scaffolds obtainable according to the methods of the present invention exhibits the base level of porosity of between 0 and 80%. The base level (smallest pore diameter) porosity as defined herein is the porosity (understood herein as volumetric porosity) of the wall structure, formed by a mesh of protein filaments, optionally with a polymeric coat or associated structure, and is preferably measured by digital optical microscopy or scanning electron microscopy (SEM), preferably digital optical microscopy.

Further preferably, the three-dimensional protein-based scaffolds obtainable according to the methods of the present invention exhibits the fibre mesh porosity of 40-80%. The fibre mesh porosity as defined herein is the dimension of gaps between the fibre array which forms the lamellae of each higher level pore structure and is preferably measured by digital optical microscopy.

Mechanical strength is vital for the survival of a scaffold, not only to withstand gross movement and external impact, but also to counter contractile forces exerted by cells within the scaffold. Cell-mediated contraction can lead to shrinkage of the scaffold that may alter the structure, and will also prevent the scaffold from creating an effective seal of the wound bed. Preferably, the three dimensional protein based tissue scaffold of the present invention has an ultimate tensile strength (UTS) of between 0.01 to 100 MPa. Preferably the three dimensional protein based tissue scaffold of the present invention has UTS of between 1-20 MPa. Even more preferably, the three dimensional protein based tissue scaffold of the present invention has UTS of between 10-16 MPa. The three-dimensional protein-based tissue scaffold of the invention has a Young's modulus of between 0.01 to 100 MPa, preferably 0.5 to 3 MPa, more preferably 1 to 2 MPa.

The three-dimensional protein-based scaffold of the present invention preferably comprises fibrin. Fibrin composite materials can also be used, for example selected from fibrin-agarose, fibrin-collagen, fibrin-alginate, fibrin-PCL, fibrin-PLLA-PLGA, fibrin-polyurethane and fibrin-PVA.

Preferably, the protein-based three-dimensional scaffold obtainable according to the method for manufacturing a protein-based three-dimensional tissue scaffold of the present invention, further comprises a non-polypeptide polymer. Preferably, the non-polypeptide polymer is selected from polymers with established biocompatibility such as hyaluronan, chondroitin sulphate, heparin heparan sulphate, chitosan, alginate, and polyvinyl alcohol. Herein, biocompatibility is understood as the possibility of using material in contact with the living tissue, substantially without any adverse effects to the tissue for certain time, for example for at least one month. Such co-polymers allow for the possibility of varying the structural, physiochemical, biochemical and consequent biological properties of the scaffold. Synthetic polymers, particularly those with proven biocompatibility and regulatory approval such as PLLA, PGA, PCL and polyanhydrides, can also optionally be used in the scaffolds of the present invention.

In a further embodiment, the present invention relates to a method of producing a cell culture substrate. The cell culture substrate is understood herein as the scaffold obtainable according to the methods of the present invention, that is deposited in a custom way. The method of producing a cell culture substrate of the present invention comprises a step wherein the protein-based three dimensional scaffold of the present invention is deposited using additive manufacturing techniques. These techniques are also referred to as 3D-printing techniques and are known to the skilled person. Preferably, a premixed HIPE including at least one protein capable of triggered polymerization, can be extruded into a coagulation bath. For example a native collagen, dispersed into a HIPE, such as a decane emulsified with 0.1-1% surfactant such as Triton-CG110, mixed and incubated at 4° C. within the printer, is extruded onto warm platform or bath (i.e. at the temperature of at least 30° C.), containing cross-linking agent. In another embodiment, a pre-mixed HIPE with an aqueous protein in soluble form, can be supplemented with a coagulation agent upstream from the extrusion nozzle within an in-line mixing chamber in a dual feed setup.

In again a further embodiment, the present invention relates to a use of the protein-based three-dimensional scaffold of the present invention in an in vitro tissue culture. The term tissue culture as understood herein relates to all the method of growing, culturing, maintaining and/or propagating cells, herein preferably mammalian cells, more preferably human cells, outside of their parent organism with the help of artificial medium comprising, among others, necessary nutrients. The methods of tissue culture are known to the skilled person. In certain cases a tissue culture substrate can be used in tissue culture. A tissue culture substrate, or cell culture substrate, as understood herein is an environment where cells that are being cultured can live, grow, and/or propagate. It can for example be a surface, or a three-dimensional environment. Within the scope of the present invention, a protein-based three-dimensional scaffold of the present invention can be used in in vitro tissue culture as tissue culture substrate.

The protein-based three-dimensional scaffold of the present invention is useful in an in vitro tissue engineering. In vitro tissue engineering encompasses broadly all methods of improving a tissue sample or replacing an indigenous tissue with another tissue sample. For the latter application, a three-dimensional protein-based scaffold of the present invention can be used.

Tissue engineering as understood herein may in certain embodiments comprise preparation of a myocardial patch. In a particular embodiment, the present invention relates to a use of the protein-based three-dimensional scaffold of the present invention in an in vitro tissue engineering, wherein in vitro tissue engineering comprises preparation of a myocardial patch. Herein, myocardial patch (also referred to as cardiac patch) is a piece of heart tissue prepared according to the methods of in vitro tissue culture and/or in vitro tissue engineering, that is to be used in a heart repair surgery, as a tissue-regenerative patch. Said piece of heart tissue is preferably grown using the three-dimensional protein-based tissue scaffold of the present invention. However, the methods of the present invention are not limited to growing/engineering heart tissue for heart repair surgery. Any tissue can be grown by using the three-dimensional protein-based tissue scaffold of the present invention. Therefore, the present invention further encompasses the use of the three dimensional protein-based scaffold of the present invention for the creation of in vitro tissue-engineered tissues, in which cells are seeded into or onto the scaffold, in aseptic conditions, in a physiological cell culture medium, and supported in an environment which allows cells to organise on or within the scaffold, to form a tissue structure or organoid or organ-like structure. These are commonly referred to as tissue-engineered constructs, tissue equivalents or skin equivalents. Such tissue engineered constructs may be used for implantation as advanced therapy medicinal products (ATMPs), or used for non-clinical investigational purposes, such as drug screening or therapy evaluation. It is noted that for such purpose, the steps of the method for manufacture of the three-dimensional protein-based tissue scaffold of the present invention are preferably performed in sterile (aseptic) conditions.

In a further embodiment, the present invention relates to the protein-based three-dimensional scaffold of the present invention for use in therapy. It is understood herein that the protein-based three-dimensional scaffold of the present invention is preferably used in a therapy of a human subject. It is in such embodiments therefore preferred, that the at least one protein capable of triggered polymerization used within the method of the present invention is of human origin, or at least is humanized. Preferably, the at least one protein capable of triggered polymerization is human fibrinogen. However, in certain embodiments of the present invention, the protein-based three-dimensional scaffold can be used in therapy of any mammalian subject, including domestic animals. It is noted that application of scaffolds made using pooled human fibrinogen as the at least one protein capable of triggered polymerization are tolerated in porcine full-thickness wounds without signs of a specific immune reaction.

In a particular embodiment, the three-dimensional present invention relates to the protein-based three-dimensional tissue scaffold of the present invention for use in therapy, wherein the protein-based three dimensional scaffold is in the micronized form for administration by injection.

In a further embodiment, the present invention relates to the protein-based three-dimensional tissue scaffold of the present invention for use in tissue reconstruction.

In a particular embodiment, the present invention relates to the protein-based three-dimensional tissue scaffold of the present invention for use in tissue reconstruction, wherein the tissue reconstruction is a dermal replacement or substitute.

In a further particular embodiment, the present invention relates to the protein-based three-dimensional tissue scaffold of the present invention for use in tissue reconstruction, wherein the tissue reconstruction is used in the treatment of a wound. The wound may be associated with tissue loss, such as, for example, a burn, a blast wound, a de-gloving injury. The wound may be a surgical resection wound, such as from a removal of a skin cancer. The wound may be a chronic wound, such as an ulcer or a pressure sore. Preferably, the protein-based three dimensional scaffold of the present invention is used in tissue reconstruction, wherein the wound is selected from a skin-loss wound, a chronic wound, a non-healing wound, a third degree burn, a fourth degree tissue loss, a traumatic skin-stripping wound and a surgical excision wound.

A surgical resection wound refers herein typically to a surgical removal of tissue using sharp dissection, over an area of skin or internal tissue of sufficient size to create a critical defect which would naturally heal by secondary intention, without intervention.

Herein burn is understood as a wound of the skin caused by high temperature. Burns are classified according to depth. Superficial burns may heal easily; partial burns are increasingly damaging with depth into the dermis. Maintaining a clean, moist wound is vital. Full thickness burns are excised to remove necrotic tissue to promote healing. Scarring is common. Burn can be considered a skin-loss wound.

Herein a degloving injury is understood as a wound caused by a shearing force to the skin edge. Skin may be removed during trauma, or may remain intact but separated from the underlying sub-dermal connective tissue. If the skin is still present, disruption to skin surface may be minimal but it appear mottled, and de-vascularisation to skin and soft tissue can be significant. This can lead to tissue necrosis, leading to infection and interrupted healing. Excision is vital before suturing.

A skin loss wound may also occur due to a crushing injury, wherein skin may not be broken, but crushing force may cause cell death. The wound may require excision or debridement to remove necrotic tissue. A wound closed with non-viable tissue left in situ will result in excessive scarring, infection and may develop into an ulcer.

Chronic wounds as understood herein include, but are not limited to, pressure sores or ulcers, diabetic ulcers, and venous ulcers. Pressure sores/ulcers (also referred to as bed sores, decubitus ulcers and ischemic ulcers) are common in subjects with limited or no mobility where skin is compressed under body weight. Regions of compressed tissue may receive insufficient blood supply, causing damage and eventually becoming necrotic. Once the epidermis is necrotised, the ulcer is susceptible to infection as supply of white blood cells is reduced by impaired vasculature. Pressure ulcers are most common on bony areas of the body—heels, ankles, hips and coccyx. Diabetic ulcers relate to a situation wherein subjects with severe or poorly managed diabetes are at risk of a subset of pressure ulcers, known as diabetic ulcers. Reduced capillary perfusion in the extremities, often coupled with atherosclerotic arteries, can lead to microangiopathy. Blood flow and perfusion are further compromised by pressure, so diabetic ulcers commonly occur on the soles of the feet. Patients with diabetic neuropathy do not feel pain from the injured region, allowing injuries to progress undetected. Venous ulcers arise from venous insufficiency, for example due to impaired venous valves that causes blood to pool in the vein. Fluid leakage from the vein into surrounding tissue can cause necrosis and lead to ulceration. Ulcers typically occur on the lower leg or ankle. Venous ulcers are the most common form of leg ulcers (70-90%).

Scaffolds may be applied acellularly, relying on native cells to migrate in and colonise, and to form blood capillaries. Scaffolds may also be pre-populated with relevant cell types (such as fibroblasts, keratinocytes, or stem cells capable of differentiating into these cells, or modulating the host inflammatory and immune responses to promote healing). There is evidence to suggest that implanting cellularised scaffolds may result in improved or accelerated healing. The improved wound healing may be preferably achieved in either cellularised or acellular protein-based three-dimensional tissue scaffolds of the present invention, wherein the protein(s) are used as the main structural substance of the scaffold; and additionally by incorporating proteins, such as growth factors, to create or modulate the host response. Any such scaffold may be referred to as a physiologically active scaffold. The proteins may be adherent to the internal and external surfaces, encouraging the surrounding tissue to integrate with the scaffold, or be released in a controlled manner. This type of tissue scaffold may be used to administer therapeutic doses of drugs to the wound over a defined period. Preferably, the protein-based three-dimensional tissue scaffold of the present invention for use in tissue reconstruction, wherein the protein-based three dimensional scaffold is positioned on the wound bed as acellular material.

In a further embodiment, the present invention relates to a method of wound treatment, the method comprising positioning of the protein-based three-dimensional tissue scaffold of the present invention onto the wound bed. Preferably, the wound as understood herein is selected from a skin-loss wound, a chronic wound, a non-healing wound, a third degree burn, a fourth-degree tissue loss, a traumatic skin-stripping wound and a surgical excision wound. Preferably, the protein-based three-dimensional tissue scaffold is implanted on the wound bed as an acellular material. The method of the present invention comprises applying or implanting or positioning the three dimensional protein based tissue scaffold at a site, such as a wound, in the human or animal body. Typically, the site is an area of the human or animal body in need of tissue regeneration or repair. The introduction of the scaffold can aid regrowth of tissue at the site in which it is applied or implanted. After applying or implanting the porous protein scaffold, a dressing material is preferably applied over the site with the porous protein scaffold. The porous protein scaffold optionally is secured at the site with sutures or staples. Before applying, implanting or positioning the three dimensional protein based tissue scaffold of the present invention, the method of the present invention may involve soaking or washing the scaffold in a saline solution (i.e. a sterile saline solution) prior to application, implantation or positioning.

The three-dimensional protein-based tissue scaffold of the present invention may is useful for engineering solid organs e.g. artificial liver, heart or kidney. The three-dimensional protein-based tissue scaffold may also be used for tissue reconstruction of dermis, fascia, tendon, ligament, pericardium, periosteum and soft tissue such as fat and muscle grafts. Preferably, the protein-based three dimensional scaffold of the present invention is used in tissue reconstruction, wherein the tissue reconstruction comprises periosteum repair.

The three dimensional protein based tissue scaffolds of the present invention can be used with any cells, as required by the envisaged application of the scaffold of the present invention. In certain embodiments, the cells may be derived from skin, soft tissue or bone. Preferably, the cells may be fibroblasts, keratinocytes, melanocytes, Langerhans cells, Merkel cells, or stem cells.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples are merely illustrative of the present invention and should not be construed to limit the scope of the invention which is defined by the appended claims.

### EXAMPLES

General Experimental Procedures

Turbiscan Stability Index (TSI)

Stability was herein reported using the Turbiscan™ stability index (TSI). This non-destructive, non-invasive technique gives the stability of the sample over its whole selected height as a function of transmitted and backscattered light compared to the same measurements at the previous timepoint. Kinetic stability is built up over sequential scans. The pattern of change across the height of the sample gives a ‘fingerprint’ characteristic of the mode of destabilisation. TSI is defined in equation 1:

$$TSI = {Σ}_{i} \frac{{Σ}_{h} \vert {scan}_{i} ( h ) - {scan}_{i - 1} ( h ) \vert}{H} \qquad ( eq . 1 )$$

where scanᵢ is the backscattering value at a given timepoint, scanᵢ₋₁ is the backscattering value from the previous timepoint, h is the height of the scan and H is the total sample height from the first to the last scan. TSI is the sum of the differences between sequential scans and thus represents the stability of the sample over a defined timeframe. TSI reduces with increasing stability.

Oil Carrying Capacity

The proposed system of determining the oil fraction at which optimum emulsion stability occurs, the ‘oil carrying capacity’ (OCC) of the surfactant, is intended as a tool for surfactant selection in a system where the desired oil fraction is known. Equally, it may be used to optimise oil fraction ‘carried’ in the emulsion when a particular surfactant is required. It is a practical tool to be used in conjunction with other metrics, such as HLB, but its simplicity and broad application make it versatile and easy to implement to formulation practice.

Herein a range of commonly used surfactants used in with oil-in-water (O/W) emulsions were studied. They all lie in the HLB range 9.8-17.6. The volume fractions of the oil (internal phase) range from classical emulsions (50, 60, 70% oil by volume) to high internal phase emulsions (HIPEs; 80 and 90% oil by volume). Emulsification and stabilisation of inverse (water-in-oil) emulsions falls outside the scope of the present work. Therefore, no lower volume fractions of oil were considered; neither were lipophilic surfactants with HLB below 10 (the only exception being Triton X-45, HLB 9.8, which was included to create a full HLB series of Triton X surfactants).

Materials

Tween® (20, 40, 80), Tergitol™ (15-S-7, 15-S-9, 15-S-15), Triton™ (X45, X100, X102, X114, X165, X405; X100 reduced, X114 reduced), Brij® (58, O20), decane and MES hydrate and sodium chloride were purchased from Sigma Aldrich. Brij® 010 was obtained from Croda International.

MES/NaCl (25 mM/150 mM) was dissolved in water and adjusted to pH 7.4. This formed the aqueous phase for emulsions. All tested were performed at 37° C.

Preparation of Emulsions

Stock solutions of surfactants were prepared in 7 ml polystyrene bijou by dispersing 0.1% (v/v) surfactant in decane so that surfactant scaled with oil fraction. These stock solutions were shaken briefly to disperse the surfactants before adding the deionised water. 4 ml mixtures were prepared in triplicate for each surfactant at various oil fractions (50, 60, 70, 80, 90% v/v oil-surfactant). Each sample was vortexed for 1 minute using WhirliMixer™ (Fisherbrand) to homogenise immediately prior to stability measurements.

Turbiscan Stability Measurements

3 ml of the sample was pipetted into a borosilicate glass cuvette, taking care to maintain a clean meniscus, and analysed using Turbiscan™ LAB Analyser (Formulation, Toulouse, France) scanning every 30 seconds for a total of 15 minutes.

Turbiscan stability index (TSI) was plotted for each surfactant as a function of oil fraction in the emulsion preparation, HLB, or time. Lower TSI indicates less change in the transmitted light, or less change in particle size and position.

The oil fraction at the lowest TSI after 15 minutes was identified as the ‘oil carrying capacity’ (OCC) of that surfactant. Further oil/buffer/surfactant mixtures were prepared at 5% oil fractions either side of this minimum (i.e. an initial minimum of 70% oil fraction would be tested then at 65% and 75% oil fraction).

Refractive Index

Refractive index of the MES/NaCl buffer was measured using an Abbe 60/HR refractometer with W type prism and sodium lamp. Entering the refractive index of the aqueous and oil phases, along with the respective volume fractions in each case, to the Turbisoft software enables generation of an estimate of dispersed particle phase over time.

The refractive indices of the decane and MES/NaCl buffer were recorded as 1.409 and 1.335, respectively.

The transmission level of the continuous phase was set at 59.64 from the t=0 reading of the MES/NaCl in the Turbiscan.

Particle Sizing

Particle size was determined using Turbisoft software using optical parameters. The refractive index of both the oil and aqueous phases was measured in triplicate using the Abbe 60/HW refractometer with W prism. Transmission level was determined by scanning a continuous phase control (MES/NaCl buffer) with the Turbiscan™. Volume fraction was equal to the amount of decane (internal phase) in each emulsion, ranging from 45% to 90% v/v.

### Example 1—Stability of Emulsions

Stability was measured using the Turbiscan™ static multiple light scattering analyser and expressed using the Turbiscan stability index (TSI) which reflects the change in transmitted and backscattered light across the height of a sample over a period of time. A higher value of TSI reflects poorer stability; a lower value reflects greater stability. However, because TSI measures rate of change, emulsions which break instantly after mixing ceases can display low TSI values.

Below the HIPE regime, at 70% oil fraction, emulsions containing Brij 010 demonstrated very poor stability when mixed at 1000 rpm but significantly improved in stability from 2000 to 5000 rpm mixing speeds (Table X1). The significant change (p<0.0001) in stability between emulsions mixed at 1000 rpm and those mixed at all other speeds suggests that above 1000 rpm, the shear rate imparts sufficient energy to the system to emulsify effectively and produce an even dispersion of droplets more resistance to breakage by migration or aggregation.

When oil fraction increased to 75%, just above the HIPE transition, emulsions containing Brij O10 gradually increased in their stability from 1000 to 3000 rpm (Table X1). At 4000 rpm peak stability was achieved. Interestingly, the stability then reduced at 5000 rpm. Samples were only mixed for 1 minute in order to make effective comparison between conditions and repeats. It is therefore possible that mixing speed was sufficient to produce a stable emulsion, but mixing time was not. This is a feature of HIPE systems where the oil is emulsified gradually. The first part to emulsify is dense and sinks, making further emulsification more difficult.

TABLE X1
Turbiscan stability index (TSI) of emulsions containing
0.1% (v/v) surfactant and either 70% (sub-HIPE) or
75% (HIPE) decane (v/v) 15 mins after mixing.

| | Oil fraction (% v/v) | | | | | |
|---|---|---|---|---|---|---|
| Mixing | 70 | | | 75 | | 80 |
| speed | Brij | Triton | | Brij | Triton | Triton |
| (rpm) | O10 | X-100 | T-80/S-80* | O10 | X-100 | X-100 |
| 1000 | 11.30 | 3.189 | 6.424 | 14.24 | 3.571 | 2.483 |
| 2000 | 2.945 | 2.758 | 3.686 | 13.67 | 2.601 | 2.225 |
| 3000 | 3.013 | 8.763 | 6.146 | 8.677 | 2.723 | 2.493 |
| 4000 | 4.266 | 3.637 | 5.860 | 2.440 | 3.213 | 9.886 |
| 5000 | 2.886 | 2.705 | 2.801 | 8.341 | 2.398 | 5.128 |

Surfactant, buffer and decane were mixed in the body of a 50 ml syringe with a Caframo impeller mixer. Surfactants tested were Brij O10 (HLB 12.4); Triton X-100 (HLB 13.4); Tween 80/Span 80 (75:25) (HLB 12.33). TSI is given to 4 significant figures.

### Example 2—Particle Size

The Turbiscan LAB Analyser uses static multiple light scattering to measure particle size. The principle advantage of this instrument is the capability to analyse HIPEs up to 95% internal volume fraction without dilution. It uses data collected from a pair of sensors to record both backscattered and transmitted light at incremental heights of a sample-containing cuvette. Transmitted light is defined as the ratio of the intensity of light entering (I₀) and exiting (I) a sample medium:

$$T = \frac{I}{{I}_{o}} \qquad ( eq . 2 )$$

The backscattered light (BS) detected by the Turbiscan is inversely proportional to l*, the mean free path of a photon travelling in a dispersion:

$$BS = \frac{1}{\sqrt{{l}^{*}}} \qquad ( eq . 3 )$$

From Mie theory, l* is proportional to the mean diameter (d) and inversely proportional to the volume fraction of dispersed phase droplets (ϕ):

$${l}^{*} ( d , Φ ) = \frac{2 d}{3 Φ ( 1 - g ) {Q}_{s}} \qquad ( eq . 4 )$$

Mie theory is a solution to Maxwell's equations that, applied to emulsions, describes scattering of light by homogenous spherical particles (dispersed phase) of a different refractive index to that of the surrounding medium (continuous phase) (Acharya, 2017, https://doi.org/10.1016/B978-0-12-809732-8.00003-X). The Turbiscan software enables computation of mean particle diameter given the volume fraction and the refractive indices of both the dispersed (internal) and continuous phases.

The principles of Turbiscan measurement come from Mie Theory and Beer-Lambert law (eq. 5).

A=ε×b×c (eq. 5)

where A is the absorbance (dimensionless), E is the wavelength-dependent molar absorptivity coefficient (M⁻¹cm⁻¹), b is the path length (cm) and c is the sample concentration (M). Absorbance is related to transmitted light as follows:

A=−log₁₀ T (eq. 6)

While Mie Theory accounts for light scattering in a dispersion, Beer-Lambert law relates the absorbance and concentration of the sample medium. Together, they describe light travelling through a sample of mono-disperse spheres through an absorbent medium.

### Example 3—Stability of Emulsions

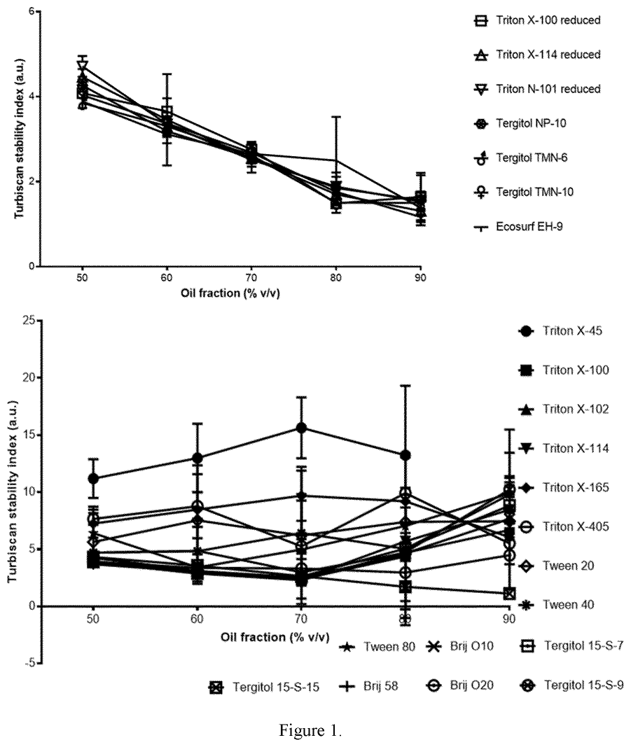

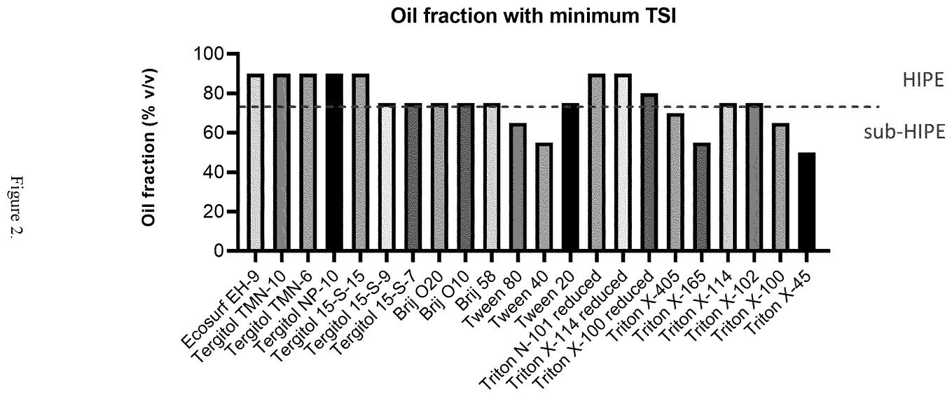

The stability of different emulsions, as measured by Turbiscan and expressed as TSI parameter, is summarized in FIG. 1. The TSI of emulsions stabilised with Triton X-(HLB 9.8) increased with each step change in oil fraction, indicating reduced stability with increased oil volume fraction. The surfactant HLB was below the required HLB of the decane oil phase (HLB 10-13) so the surfactant provided poor emulsion stability. As oil fraction increased, the surfactant was dispersed over greater surface area and stability further reduced. At 80% oil fraction, the TSI reduced—but stability also reduced. This discrepancy arises from the Turbiscan measurement principles. TSI is a kinetic measurement and relies on the differential light transmission/backscattering between each scan. Samples that decay too quickly—with the most rapid droplet deformation occurring before the first time point—register a lower TSI as a result.

The other surfactants—in the HLB range 12-17—showed a different pattern of stability with increasing oil fraction. Stability increased (lower TSI) as oil fraction increased up to a certain point, after which stability reduced, often rapidly. This behaviour is characterised by a U-shaped curve with the minimum representing the optimum oil fraction for emulsion stability with each surfactant—the “oil carrying capacity” of the surfactant.

Group B surfactants (Table 2), such as reduced Tritons and branched Tergitols, showed a very strong pattern of increased stability (reduced TSI) with increasing oil fraction that continues into the HIPE regime (P<0.0001) (FIG. 1).

The difference in stability in branched Tergitols (TMN-6, TMN-10) compared to Tergitol T-S-7, -9 and -15 suggests a structural contribution. The bulkier surfactants may provide enhanced stability at higher oil fractions by virtue of the more rigid structures reducing rotation and movement of surfactants at the interface which may make them better able to maintain the boundary between dispersed and continuous phase despite unfavourable thermodynamics.

Among Tergitols, the conventional surfactants span a wider HLB range (Table 1) than the branched variants (Table 2). Both Tergitol T-S-9 and Tergitols TMN-6 and NP-10 have HLB ˜13, matching the experimentally determined required HLB range for decane. These results show a much greater dependence on other surfactant properties, such as structure, rather than HLB.

The pattern of behaviour is different with other classes of surfactant (FIG. 1). The oil carrying capacity metric is a method of distinguishing these classes of surfactant and identifying those suitable for stabilising HIPEs as opposed to lower internal phase emulsions (oil fraction 70%).

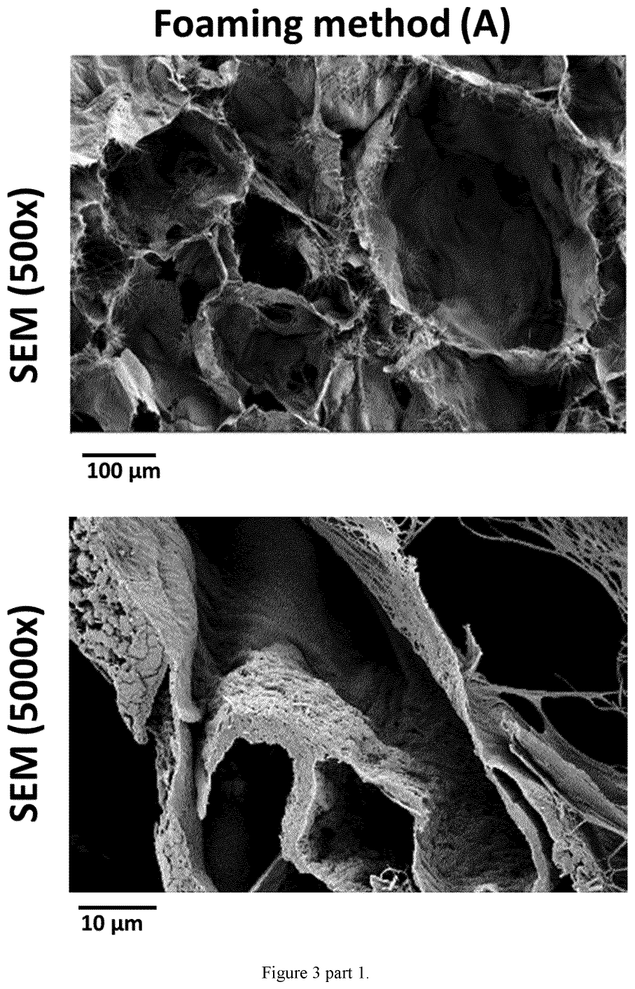

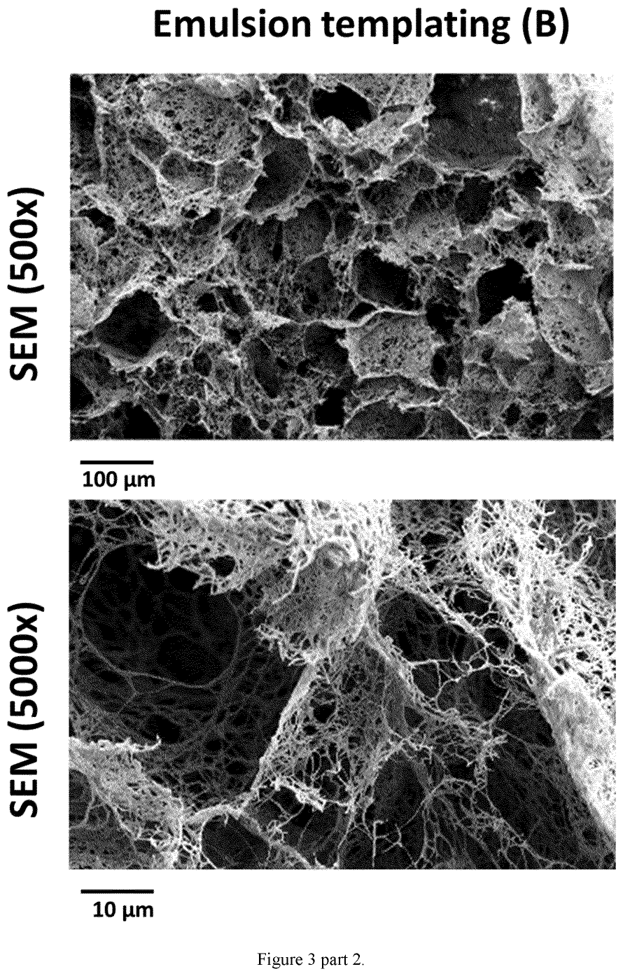

Emulsions containing Triton surfactants X-100, X-102, X-114 and X-405 all display maximum stability at 70% oil fraction (FIG. 2). With TSI<3, TX-102 and TX-114 were particularly effective at 70% v/v oil fraction, though they are less effective at higher or lower oil: aqueous compositions. The use of different Triton surfactants was highly influential in the stability of the resultant emulsion (P<0.0001). Triton X-45 shows a symmetrically opposite pattern of behaviour, arising from its low HLB, beneath the o/w emulsifying regime.

### Example 4—Manufacture of Scaffolds of the Present Invention

Emulsion Preparation

Emulsions were prepared separately for each 5×5 cm scaffold; each with a volume of 22 ml. Decane formed the oleic phase (90% v/v). The aqueous phase final composition (10% v/v) at casting comprised fibrinogen (2.5% w/v clottable protein), thrombin (2.5 IU ml⁻¹; added in a ratio of 1 part to 4 parts fibrinogen), calcium chloride 2.5 mM, and PVA (85-89% hydrolysed, average MW 130 kD, 0.25% all dissolved in a MES/NaCl buffer solution (pH 7.4). For each scaffold, 20 μl surfactant was dispersed in 20 ml decane (0.1% v:v) in a 50 ml universal tube. 5 μl calcium chloride solution (1 M), 625 μl MES/NaCl buffer and 100 μl 5% PVA solution were added and shaken vigorously until the contents emulsified fully (determined by dramatic change in viscosity and opacity). 1 ml warmed fibrinogen solution (5% w/v clottable protein) was added and the tube was shaken vigorously to disperse the protein. 250 μl warmed thrombin was added and the mixture was shaken immediately and vigorously for 30 s and immediately poured into a casting tray.

Scaffold Manufacture

Each final emulsions was poured into a square-edged casting tray (weighing boats serve as convenient for this purpose) which was tapped to remove air bubbles. The tray was covered to reduce evaporation and contamination and incubated for 1 hour at 37° C.

Scaffolds were incubated for 1 hour at room temperature with 25 ml cross-linking solution (abs. ethanol, 0.1M aqueous MES solution (pH 7.4) and glutaraldehyde (25% aqueous solution) in a ratio of 100:25:1). The scaffold was flipped over the process repeated for another hour with fresh cross-linking solution. During cross-linking the scaffolds turn from white to yellow (colour intensity is dependent on concentration of glutaraldehyde and fibrinogen present). Residual glutaraldehyde and Schiff bases were reduced by a series of 5 reducing washes using 0.1% A sodium borohydride in distilled water. The solution was changed and the scaffold flipped over after each wash. This process was repeated for a further four washes with distilled water, flipping the scaffold and replacing with fresh water each time. The scaffolds were then washed in 0.2% aqueous PVA solution (w/v) for 5 minutes each side before draining. Scaffolds were then lyophilised overnight at −40° C. under vacuum, (VirTis Genesis 25ES, SP Scientific, Warminster, Pennsylvania, USA).

Comparison of the Protein-Based Three-Dimensional Tissue Scaffold of the Present Invention and Foamed Scaffold of Similar Composition

Microstructural Evaluation

Three samples per scaffold (approximately 8×3 mm) were cut and mounted in cross-section and sputter coated on stubs. Micrographs were taken with a Zeiss Supra 55 VP scanning electron microscope (aperture 20 μm; current 5 kV). Each sample was imaged at three different sites. Void size and size distribution (including all measurable voids) was evaluated using the measure function in Image J. The images used for analysis were all 1000× magnification micrographs, as these images contain clear resolved structures of interest at both pore and interconnect level.

Pore and interconnect sizes are measured manually from SEM micrographs using Image J. The largest round shapes that can be resolved as assigned as ‘pores’; the smaller structures within these are assigned ‘interconnects’. Immeasurably small distances (using this method, at appropriate magnification to resolve pores) are designated diffusional gaps (space between fibrin fibres as opposed to templated structures) and are not recorded owing to their small size and high frequency.

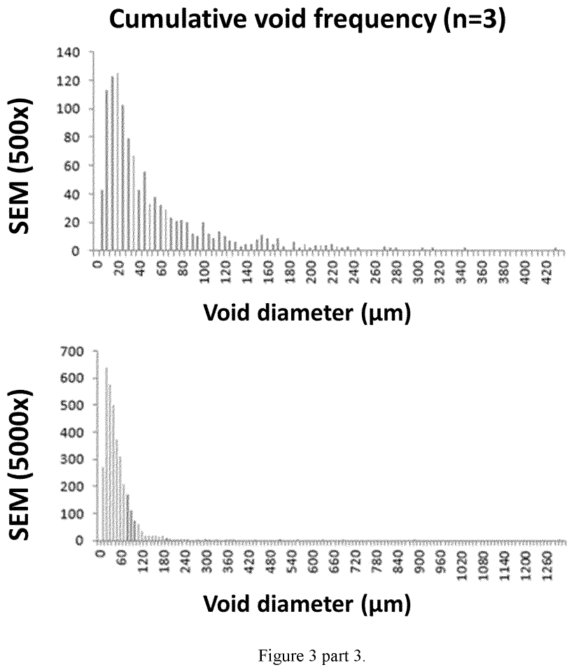

Emulsion templating offers better control over droplet size and wall thickness compared to other methods such as foaming (FIG. 3). The foaming method used for reference is as described in the patent PCT/GB2013/051158. The foamed scaffold had regions of high porosity but was very heterogeneous. The foaming method created a somewhat interconnected network between the pores generated by escaping gas. Variability in porosity and wall thickness were governed by dispersal of foaming agent. If the foaming agent is not dispersed uniformly dense regions of material form. In vivo these would provide no functionality and may exacerbate the inflammatory response and could lead to macrophage attack (reducing scaffold persistence) or fibrous encapsulation.

By contrast, emulsion templated scaffolds show a higher degree of consistency in pore structure, with large pores in the range 120-180 μm, interconnecting pores in the range μm, and a consistent fibre mesh structure setting the base-level porosity, as shown (FIG. 3).

### Example 5—Determination of Pore Structure of an Emulsion of the Present Invention

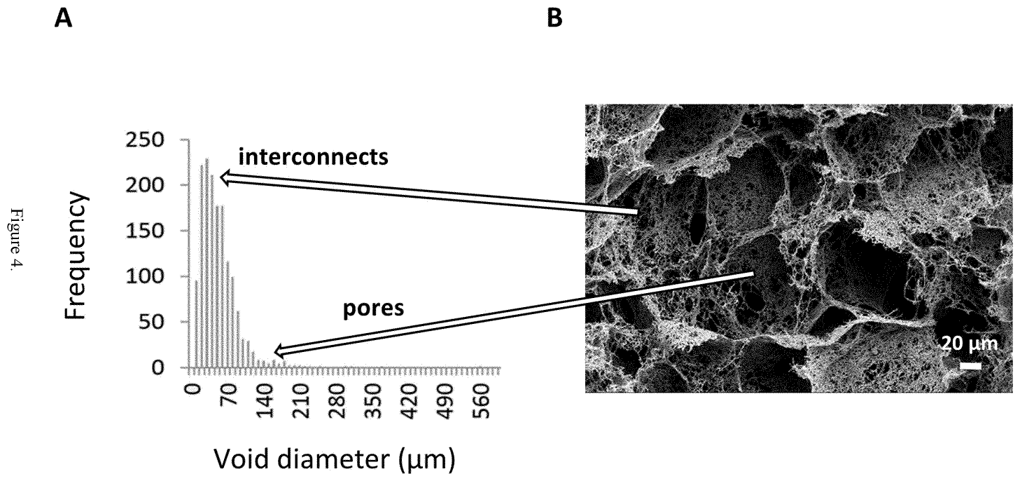

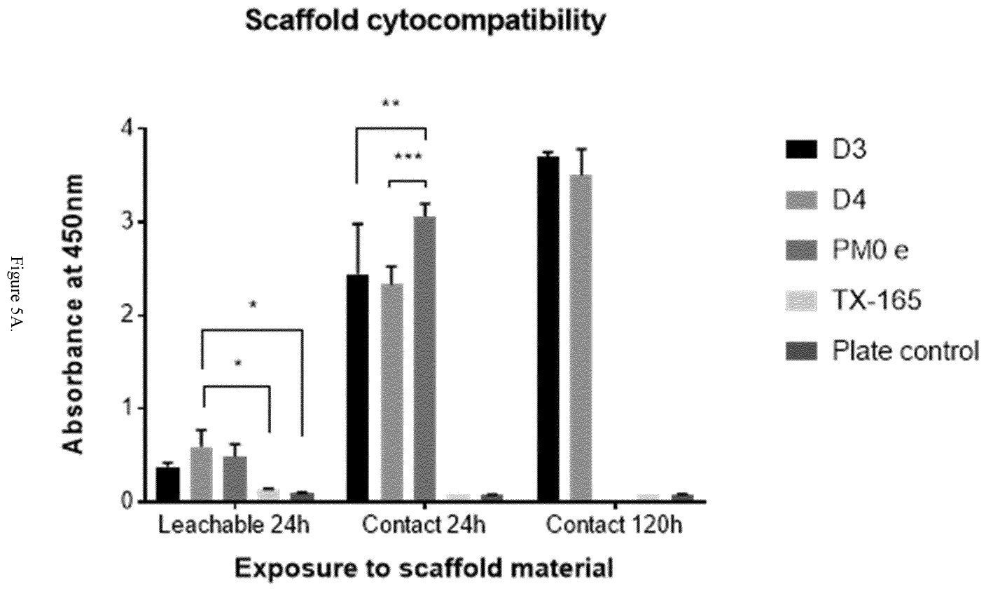

FIG. 4(A) shows cumulative frequency from three sites analysed from the same scaffold as shown in Example 4B. Mean pore size 102.35 μm, SD=74.83; mean interconnect size 35.05 μm, SD=15.34; mean diffusional void size 7.13 μm, SD=1.96. All measurable voids were quantified at the widest point using Image J (1000× micrograph). Pores were determined as the largest resolvable round structures on the image; interconnects are the smaller gaps within them. Very small gaps resulting from spaces between fibrin fibres, as opposed to template shape, were designated as the base level of porosity (wall porosity). Only the larger diffusional gaps were quantified, as most are too small to measure accurately by this method. FIG. 4(B) shows a typical cross-section of a freeze-dried scaffold (Zeiss Supra 55 VP; 5 kV, 1000× magnification).

### Example 6—Cyto-Compatibility

Cylindrical samples were cut with an 8 mm biopsy punch (n=3) were cut from two scaffolds. These were washed twice with 70% EtOH, twice with PBS and once with DMEM. GFP-MSC-hTERT* were seeded (p12, 50,000 cells/well) onto the hydrated scaffolds and a plate control with 150 μl DMEM+FBS+PS. At 24 h (acute response time) and 120 h (chronic response time), 300 μl DMEM/CCK-8 (10:1) was added and incubated for 4 hours at 37° C. before reading absorbance at 450 nm.

Two scaffolds of the same composition (D3, D4) from different manufacturing batches (0.1% Tx-165 surfactant, 90% porous, 2:1 fibrin: PVA, 2% fibrinogen) were tested with triplicate samples from each. A scaffold with the same wall material and comparable porosity and pore size, manufactured via a foaming method, was used as a 3D control. This material is known to be non-cytotoxic, verified with both in vitro and in vivo assays. Surfactant (in this case, Triton X-165) was added to the same volume of media to give a concentration of 0.1% v/v and was used to test inherent surfactant cytotoxicty.

At 24 hours in culture, cell count (h-tert human mesenchymal stem cells) was comparable between the two scaffolds but ⅗ the count on the 2D plate control. 24 h was selected as a time-point relevant for acute toxicity. However, by 120 h cell count on the scaffold had increased to be comparable with the 2D plate control. This would suggest there was no toxic response as cell numbers would not be expected to recover to such a degree. It is possible that the reduced cell population in the scaffolds at 24 h is due to the cells expending energy exploring and colonising the 3D environment rather than proliferating.

The difference in absorbance between the templated scaffolds (D3, D4) and the foamed scaffold (PMO e) in the 24 h leachable assay was not significant.

In the contact assay, there was no significant difference between the two templated scaffolds at either 24 or 120 h. However, the reduced absorbance of the templated scaffolds compared to the foamed scaffold was significant at 24 h (D3*; D4**). The difference in absorbance between the unbound surfactant and all scaffolds was highly significant (****), suggesting that the surfactant used for templating, although cytotoxic in itself, is largely removed during washing and poses low risk of cytotoxicity during application. This initial result suggests no cyto-toxic response at either acute (24 h) or chronic (120 h) exposure time.

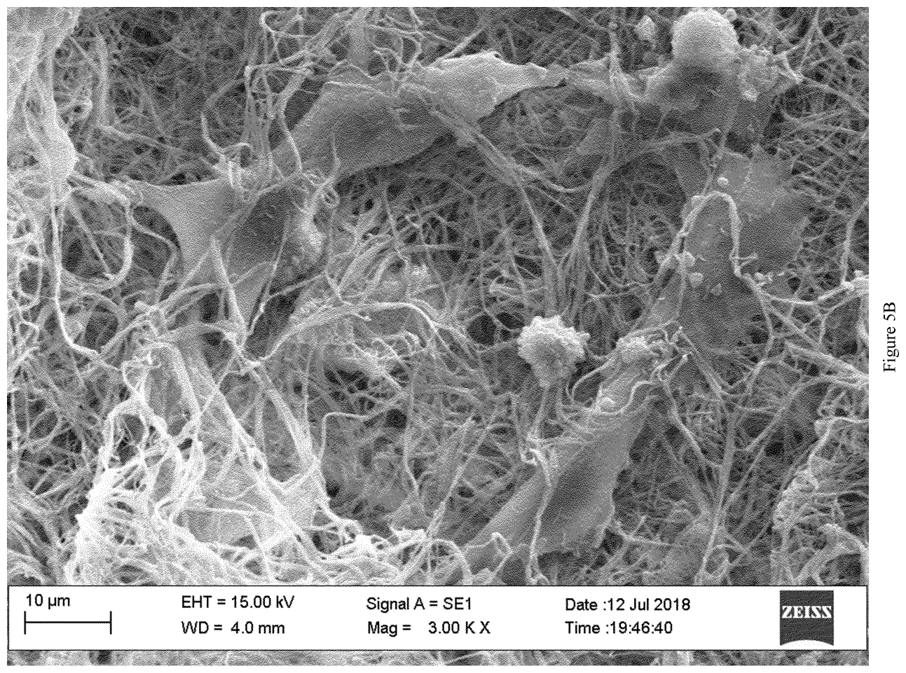

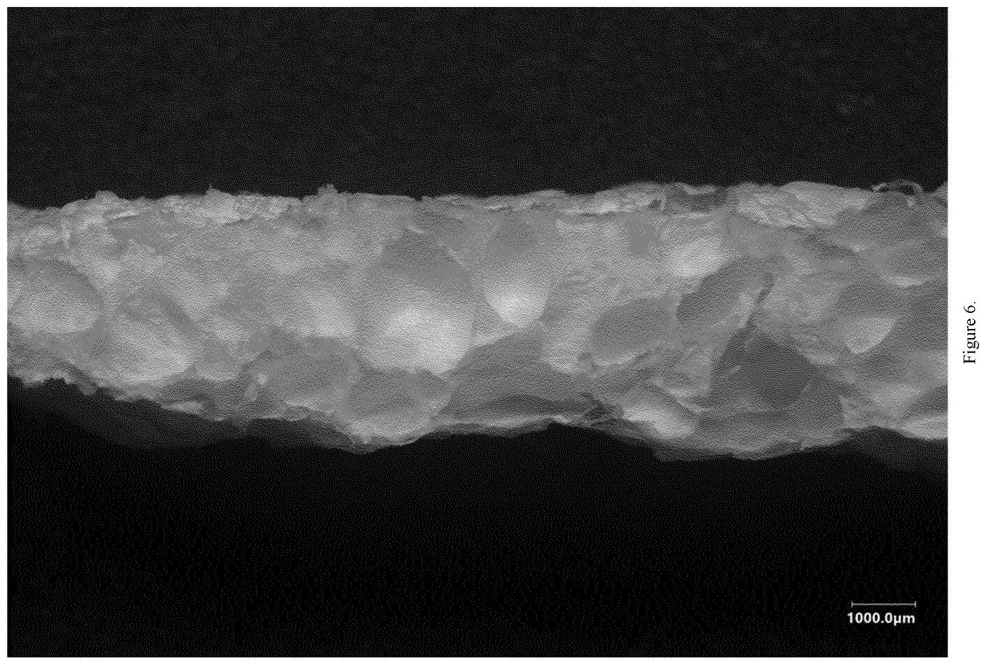

The results are summarized in FIG. 5. This initial result suggests no cyto-toxic response at either acute (24 h) or chronic (120 h) exposure time.

### Example 7—Macro-Level Pore Structure

Digital imaging analysis of macro-level pore structure of scaffold of Example 4, showing a range of pores from base level around 100 um up to 1000 um, resulting from coalescence of oil drops.

### Example 8—Effect of Fibrinogen Concentration on Scaffold

Scaffolds were prepared as described for example 4, except that two concentrations of protein were used (10% and 5%), with ratios of thrombin and calcium varying in proportion.

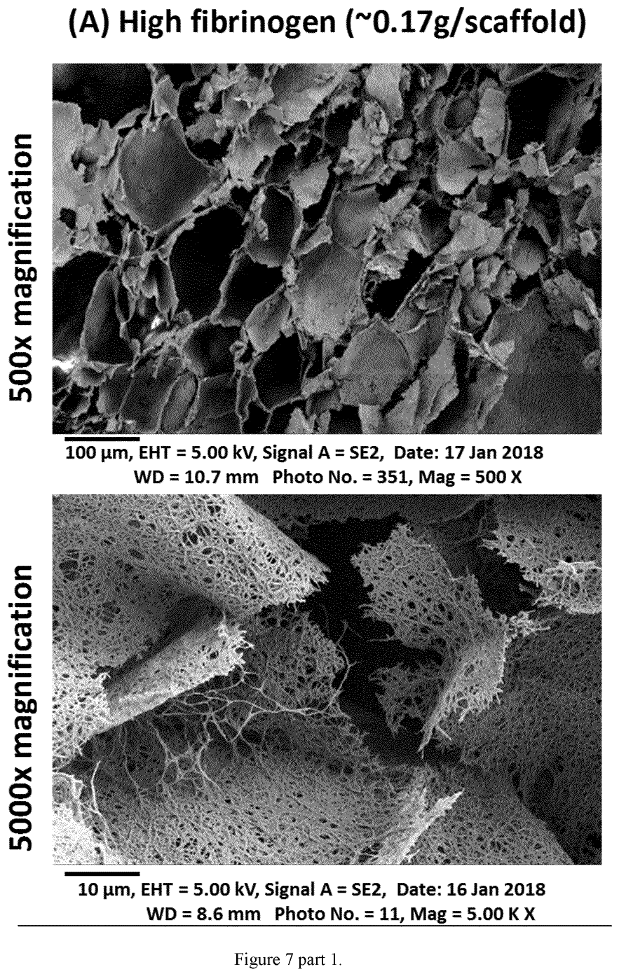

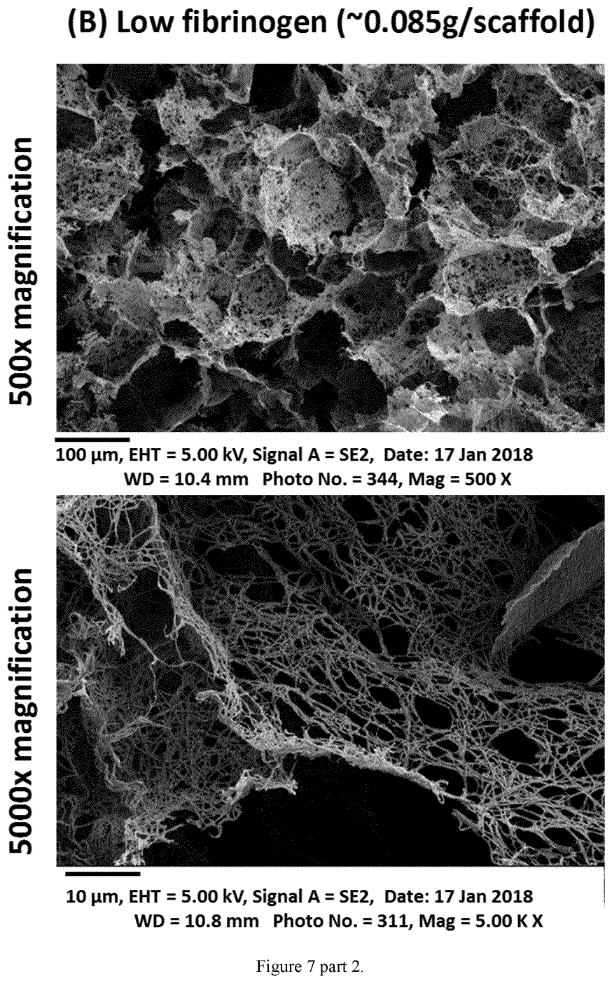

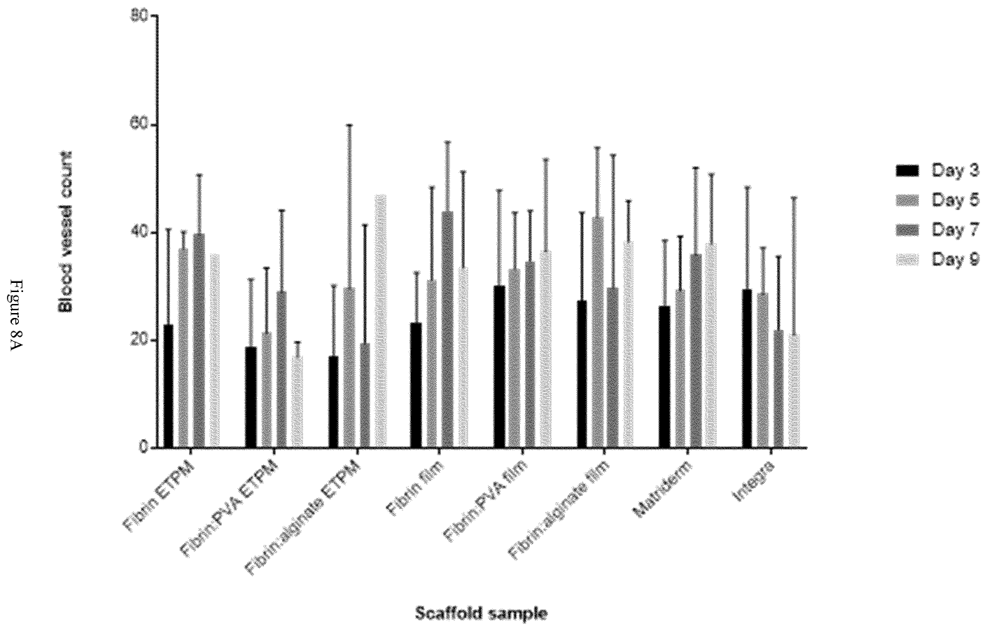

The precursor emulsion must contain sufficient fibrinogen to form fibrous networks around the largest of the pores that can adequately support their own weight when the template is removed. The fibrinogen volume is limited by the total emulsion volume (i.e. the size of scaffold desired) and the volume fraction occupied by oil (i.e. the desired scaffold porosity) as well as any other materials to be included. Fibrinogen solutions become extremely viscous above 5% (w/v, clottable protein). 10% was found to be the highest useful concentration with the method and equipment described here. The concentration of fibrinogen in the aqueous phase of the HIPE was therefore studied at 5% and 10% w/v clottable protein, maintaining proportions of thrombin and calcium chloride, to produce dense (FIG. 7(A)) or more delicate structures (FIG. 7(B)). Reducing the protein concentration resulted in thinner yet more robust scaffolds with excellent handling properties (wet and dry) as well as increased permittivity and interconnectivity as illustrated in the cross-sectional SEM images.

A higher fibrinogen concentration ensured more fibrin coverage around the oil droplets and better mechanical strength. Lower fibrinogen concentration gave a more diffusive, fibrous basket-weave structure which in turn gave better diffusivity, permeability and pore interconnectivity These features are all important scaffold design considerations so fibrinogen content must be modulated to achieve the optimum balance.

The high-fibrinogen scaffold was easier to manufacture owing to the improved mechanical strength. Fibrin formed rapidly and was therefore more forgiving of emulsion instability. Template fidelity was good, with tightly packed pores of similar sizes in the region 50-200 μm. However, the walls were very dense, resulting in reduced interconnectivity and permeability. Additionally, with the fibrin fibres are so closely associated, the PVA co-polymer tended to cover groups of fibres rather than individual strands and further closed up the diffusional gaps (FIG. 7(A), 5000×).

When fibrinogen concentration was too low, the scaffolds collapsed during manufacture, suggesting the fibrin was insufficient to surround larger pores. These are not pictured as they could not be preserved through to drying. Reducing the fibrinogen concentration from the high concentration (10% w/v clottable protein) to 5% (final 2.5%) w/v produced a scaffold which was readily handleable throughout manufacture. Template fidelity was as good as in the high-fibrinogen scaffolds, with tightly packed rounded pores. The sparser distribution of protein around the interface resulted in a more open structure with much greater interconnectivity and more diffusional gaps for the same meso-porosity. Under higher magnification, individual fibrin fibres were clearly visible (FIG. 7(B)). These were sufficiently closely arranged to impart strength, but well-spaced enough that the PVA co-polymer did not coat them and seal up the smallest voids.

### Example 9—CAM (Chorio-Allantoic Membrane) Assay

Method

On receipt, eggs were checked for hairline fractures, mechanically cleaned with dry paper towel and refrigerated until required (up to one week). Before use the eggs were cleaned with chlorhexidine gel and dried thoroughly before weighing. They were then positioned in the pre-conditioning incubators on their longitudinal axes and the upper surface marked.

Briefly, fertilised eggs were incubated at 37.5° C., 60-62% humidity with the incubator (RCom KingSuro Max 20, Autoelex Co. Ltd., Juchon, Korea) rotating through 60° every hour. (Eggs may be stored at 4° C. for up to 7 days prior to commencing the experiment). After 72 hours each egg in turn was removed from the incubator and gently broken into an octagonal-based weighing boat (cleaned with 70% ethanol), and lidded with a second inverted weighboat, and transferred in a larger tray on porous pads wetted with sterile water, to a humidified incubator (37° C., 80-85% humidity (HERAcell VIOS 160i, Thermo Scientific). After overnight incubation, any non-viable embryos, determined by absence of heartbeat were removed and destroyed.

All scaffolds and films were washed in 70% ethanol then three times in sterile PBS, and were equilibrated in fresh, sterile PBS at room temperature. Circular samples were cut with a sterile 8 mm biopsy punch four scaffolds per egg were positioned at the edge of the capillary plexus, equally spaced around the circumference according to a pre-determined matrix. Two samples from each scaffold, with three scaffolds per material condition were used. Each sample and egg were photographed after placement. Eggs were then returned to the incubator for a further 8 days and were checked daily for viability, with daily refreshing of the humid trays with fresh water daily, or as required, to maintain overall incubator humidity of 80-85%. Each egg was removed and photographed on days 1, 3, 5, 7 and 9. At each timepoint, the embryo and each sample were imaged at 7.5× magnification with a camera attachment (GXCAM-9, GT Vision Ltd. Stansfield, UK; Nikon SMZ745T stereomicroscope).

Early on day 9 after imaging, all accessible samples were gently excised with forceps, using a scalpel to remove excess albumen where necessary. Each sample was deposited into the well of a 24 well plate. Embryos were destroyed immediately after sample excision.

The haem content of the excised scaffolds was assessed by Drabkin's assay, using the protocol provided by the manufacturer (Sigma Aldrich, available online). Samples were not treated with anticoagulants or calibrated to take account for lipids, abnormal plasma proteins or erythrocyte stoma. Briefly, excised scaffolds were placed in 24-well plates and incubated with 2 ml Drabkin's reagent for 30 minutes at room temperature. Absorbance on the spectrophotometer was set to 0 using distilled water as the reference. The samples were removed before measuring the absorbance of each well at 540 nm. A standard curve was prepared using reconstituted human haemoglobin (100 mg/ml to 6.25 mg/ml).

Angiogenic potential of materials was assessed by counting blood vessels penetrating the samples on each imaging day using Image J analysis of light micrographs.

Samples Tested:

Emulsion templated scaffolds Fibrin, Fibrin PVA, Fibrin/Alginate non-porous film Fibrin Fibrin-PVA Fibrin-alginate Reference materials: Matriderm and Integra (silicone layer removed)

Results and Discussion

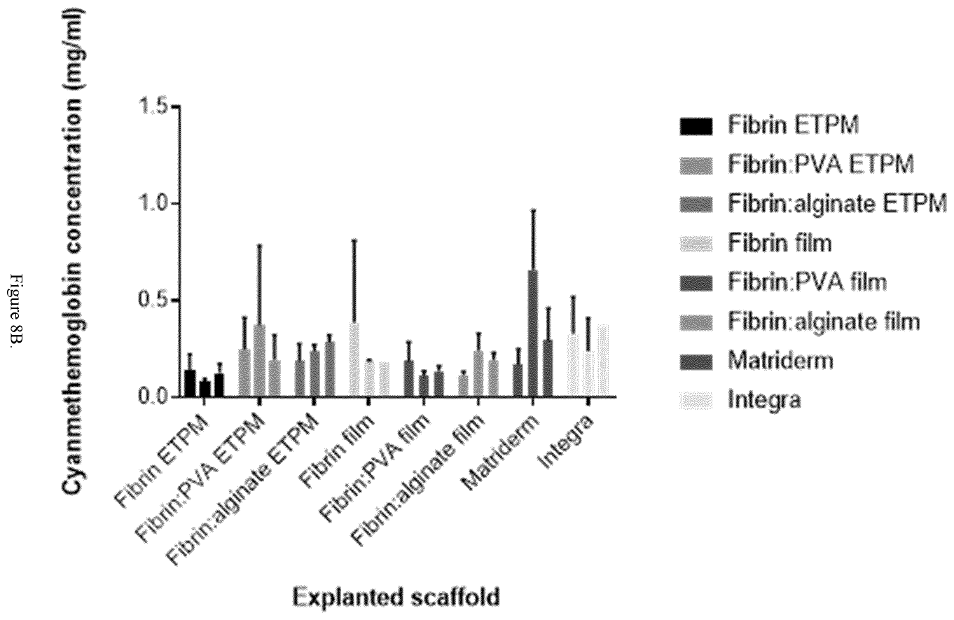

Most test materials perform comparably with the commercial comparators Matriderm™ and Integra® (FIGS. 8A & B). The 90% fibrin templated scaffold performed particularly strongly, with greater blood vessel interaction than either commercial product on assay days 5, 7 and 9 (incubation days 9, 11 and 13). Prior to assay day 5, the capillary network in all cases was still growing; from day 5 onwards the entire surface of the culture dishes were vascularised and the capillaries moved from exploration and expansion to consolidation and maturation. In general, all samples showed increased blood vessel interaction over time. (Some reductions of vessel counts at a single timepoint are likely to have arisen from the difficulty ascertaining which blood vessels were truly interacting with the samples, or due to conformational changes in the capillaries over time, bending close to the edge of sample discs.)

Notably, Integra® was the only material to show consistent reduction in the number of interacting blood vessels across each time point. At day 3, the number of interacting blood vessels was the highest of all samples but this declined over the course of the assay. The remaining blood vessels in the vicinity did not appear to have increased in diameter, suggesting the reduction in number was not offset by maturation of the major vessels.

In contrast, the number of blood vessels interacting with Matriderm™ samples increased day-on-day with the greatest increase occurring between days 5 and 7 after sample placement. This time point is of interest as it marks the change from exploratory blood vessel growth over the air interface to maturation. An increase in blood vessel interaction from day 5 onwards is therefore more likely to be indicative of a material influence rather than random growth.

Among the fibrin-based materials, there was no significant difference in the number of blood vessels counted either between the same materials at different timepoints, or different materials at the same timepoint. No advantage of any one material over another was apparent. The fibrin-only scaffold showed increased blood vessel interaction compared to fibrin-PVA and fibrin-alginate scaffolds, but the difference was not statistically significant. There was no discernible difference in the performance between the three different fibrin-based films.

The fibrin films showed a greater number of blood vessels interacting compared to their 3-dimensional scaffold counterparts. However, this number is likely to be misleading as the films were more transparent and allowed greater visualisation, making it difficult to separate interacting vessels with non-interacting blood vessels below the sample. The results cannot therefore separate between material and structural contribution to angiogenic potential. Secondly, the pro-angiogenic mechanism of fibrin is likely to require initial interactions of either leukocytes or endothelial progenitor cells, and the CAM membrane may present a cellular epithelial barrier to such direct interactions.

While the comparative results are not compelling, the established biocompatibility of both Matriderm™ and Integra® and similarity of results suggest that an important finding from these results is that the test materials show excellent biocompatibility and an absence of inflammatory response or proteolytic degradation.

The results of the haemoglobin assay FIG. 8B and the number of interacting blood vessels in FIG. 8A are somewhat discrepant. The Matriderm™ samples had among the highest number of interacting blood vessels at day 9, were in broad agreement with the Drabkin's assay. However, the fibrin ETPM scaffolds, fibrin-PVA films and fibrin-alginate films which displayed similarly high levels of blood vessel interaction in the CAM assay were found to contain the lowest levels of haem by Drabkin's quantification (<0.07 mg/ml). This may be due to the difficulty in extracting the samples cleanly from the albumen, and because the fibrin-based scaffolds and films are all highly elastic in behaviour, as are blood vessels, and the albumen ‘matrix’ was highly viscous by the end of culture. There was resistance from all three components during sample harvesting. It is also possible that many of the blood vessels observed interacting with samples, particularly with the transparent films, were passing adjacent and below the samples. Films were retrieved largely intact without signs of tearing, which supports this supposition.

Although the fibrin-PVA and fibrin-alginate scaffolds showed reduced numbers of interacting blood vessels, the mean haem detected was greater than in fibrin only scaffold samples. The addition of PVA, particularly, has been shown to significantly improve the mechanical properties of the fibrin-based scaffolds. It is difficult to draw positive conclusions from the results, but they do not show any detrimental angiogenic effect when PVA or alginate are used to augment the mechanical properties of the fibrin.

### Example 10—Mechanical Testing

Cross-linking provides additional strength and stability to protein scaffolds, making intrinsically delicate structures closer in strength to native skin tissue, more robust to the proteolytic wound environment, and easier to handle on placement. It is widely reported that cells can sense and respond to the substrate stiffness. Additionally, the mechanical properties of a scaffold can influence both stem cell differentiation and morphology, so it is important to match closely with native tissue (Freed et al., 2009; Hollister et al., 2002; Hutmacher, 2001). Matching scaffold strength to the native tissue also helps promote integration, as the implant will behave in a more contiguous manner. Cross-linking also increases resistance to proteolytic degradation so the scaffold can persist in vivo for a clinically useful timeframe.

In vivo, cross-linking of newly-formed soluble fibrin fibrils by activated coagulation factor XIII stabilises the fibrils to form a strong, elastic insoluble clot (Standeven et al., 2005). The same effect may be achieved economically in artificial fibrin constructs by chemical cross-linking with reagents such as genipin (Dare et al., 2009; Linnes et al., 2007a), glutaraldehyde (McManus et al., 2006) or carbodiimides such as EDC with NHS (Grasman et al., 2012); or by photo crosslinking, such as with the aid of a ruthenium catalyst (Bjork et al., 2011).

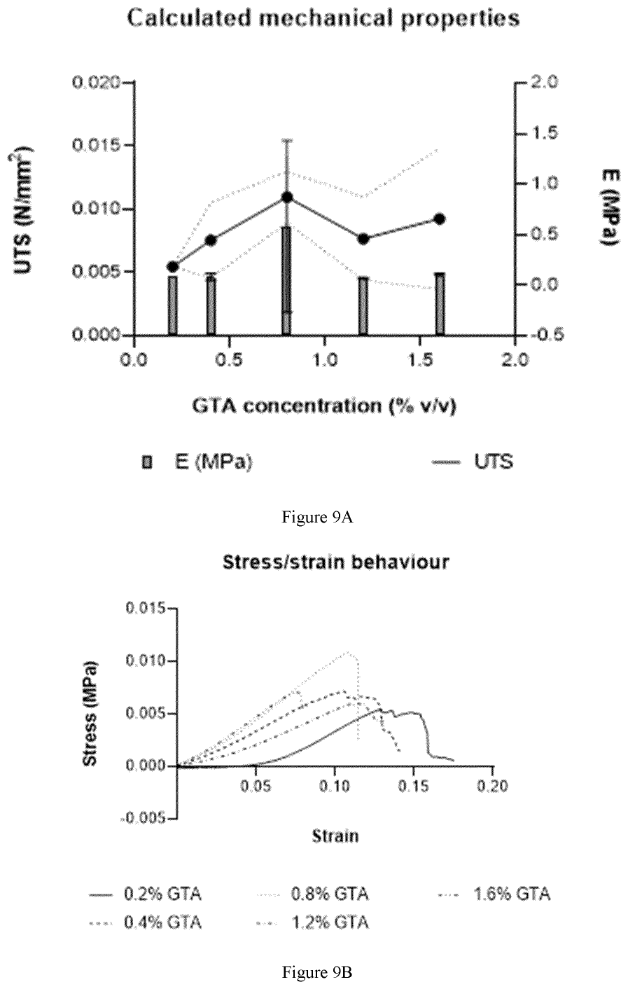

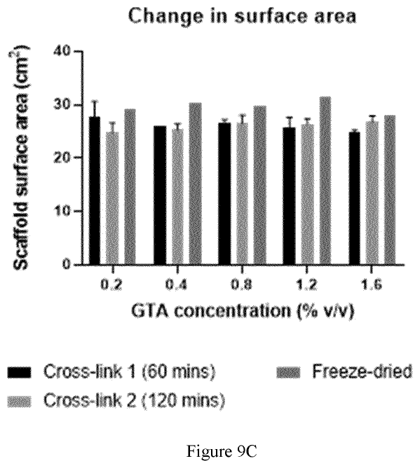

It was expected that increasing glutaraldehyde concentration would increase Young's modulus (and ultimate tensile strength in line with this as the material is elastic). However, there was little difference in modulus between 0.2% and 0.4% v/v glutaraldehyde. 0.8% v/v produces significantly higher Young's modulus, but higher concentrations showed modulus reducing to very low values. UTS showed a slightly different trend, but in both cases 0.8% glutaraldehyde concentration was optimal (FIG. 9A).

Cross-linking creates shorter length bonds between fibrin molecules, resulting in contraction of the bulk scaffold. It was hypothesised that more cross-linker would result in more contraction, so scaffolds were measured along the x and y axes after the first and second cross-linker washes and then again after the scaffolds were reduced, washed and freeze-dried. Generally, the scaffolds did contract between the first and second cross-link.

The contraction from ‘as cast’ (5×5 cm) reduced after freeze drying to a very small decrease. It is possible that the reduction in the hydrated state is not due to cross-linker so much as hydrostatic pressure from the surrounding solution. Scaffold shrinkage showed no dependence on cross-linker concentration except at 1.6%, and reduction in the final dried scaffold was minimal (FIG. 9(B)).

Tensile testing of scaffolds cross-linked with 0.8% glutaraldehyde showed elastic behaviour to failure (indicated by the straight ascending slopes on the graph). All three repeats failed at the same strain (˜1.7). Stress varied between sample, with the first replicate exhibiting more soft behaviour than the second, but these were broadly in agreement (FIG. 9(A)). This could suggest that the lowest glutaraldehyde concentration is sufficient to crosslink the fibrin, or that most of the mechanical strength comes from the fibrin and not the additional crosslinking. The tension imparted from the HIPE structure itself—where the interfaces may be approximated as I-beams, or a classical honeycomb structure—may also contribute to mechanical strength as reduce the role played by glutaraldehyde crosslinking (Kravchenko et al., 2018).

### Example 11—Addition of PVA to Enhance Elastic Behaviour

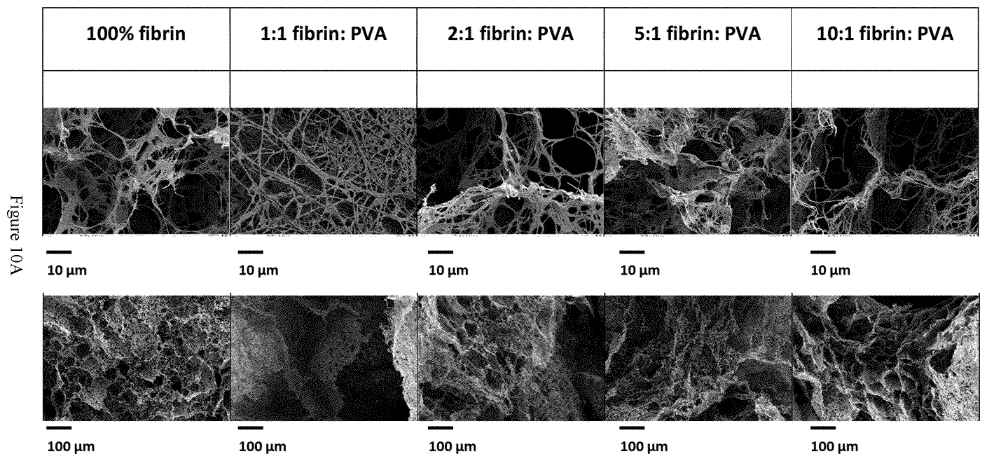

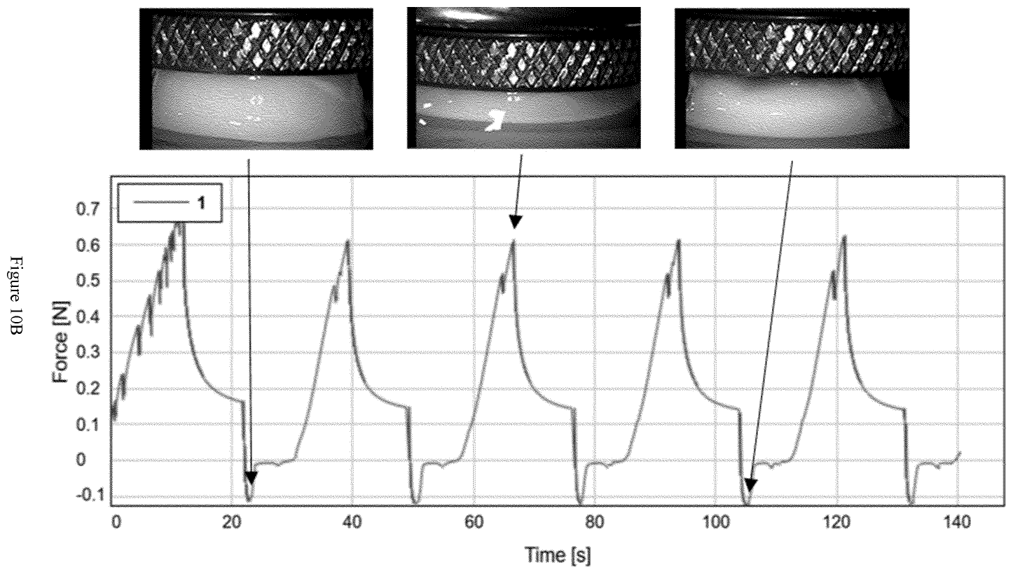

FIG. 10A shows the effect of varying the proportion of an aqueous polymer PVA, added with fibrinogen (5% w/v clottable protein), to give the w/v % ratios indicated as in method described in example 4, on the base level of resultant fibrin fibre organisation and pore structure. Scaffolds imaged by SEM, as described. FIG. 10B shows the effect of addition of PVA to a bulk fibrin gel at ratio of 2:1, on conferring elastic behaviour.

Native fibrous fibrin produces scaffolds with optimal microstructure, but they behave in a brittle manner at macro-scale and can be fragile to handle. Scaffolds must be sufficiently robust to withstand manufacture, and to be cut, positioned and stapled or sutured into place in clinical use. PVA was incorporated as a co-polymer to increase bulk elasticity of the scaffold. As previously described, aqueous component volumes are limited by the desired porosity and associated oil fraction, so a working solution of 5% w/v PVA was used. At 1:1 fibrin: PVA, the PVA was found to coat closely arranged fibrin fibres and draw them into thick bundles. While this did improve elasticity and handleability, it disrupted the microstructure by reducing nano-texturing and diffusional gaps. At meso-scale, reduced interconnectivity and diffusivity were evident (FIG. 10A).

Halving the PVA ratio to 2:1 gave a comparable improvement in elasticity but with less detriment to the microstructure. Although there was some association of fibrin fibres, diffusional gaps and interconnects remained evident. The structurally and biologically relevant hierarchy remained but the mechanical behaviour was much improved, so the 2:1 composition was deemed optimal.

At 5:1 and 10:1 fibrin: PVA, the microstructure was not significantly affected by the addition of PVA. However, the macro-scale mechanical properties were similarly unaffected so there was no benefit of PVA addition at these levels. 2:1 fibrin: PVA represents a concentration of 1% w/v PVA in the emulsion mixture. Even at this level, elasticity of the scaffold was increased.

Bulk cross-linked hydrogels of 2:1 fibrin: PVA, at 1% fibrin: 0.5% PVA, in discs of approximately 10 mm×3 mm, were tested in their hydrated state under cyclic compression. The 2:1 material showed excellent recovery between each cycle, with closely similar stress-strain and recovery curves being produced each time (FIG. 10B). The linear slope on the ascending side is characteristic of elastic behaviour. The descending slope of each cycle represents the viscous component and is likely associated with the displacement and re-association of water with the hydrogel. The displaced water is visible in the second image of the compressed gel, and just visible drawing up the left edge of the right-hand image where compressive force has been released.

Statistical Analysis

In the examples discussed herein, unless otherwise stated the statistical analysis has been performed as follows. Statistical analysis was performed using GraphPad Prism version 8.1.2. Significance was evaluated using one- or two-way analysis of variance (ANOVA), as appropriate, with Tukey's post-hoc multiple comparison tests. Levels of significance were assigned as follows—* where p<0.05; ** where p<0.01; *** where p<0.001 and **** where p<0.0001.

### Example 12—Ability of Nano-Fibrous Scaffolds, which were Subsequently Stabilised by Covalent Cross-Linking, to Support Proliferation of Human Cells Relevant to Tissue Reconstruction

The ability of nano-fibrous scaffolds, which were subsequently stabilised by covalent cross-linking, to support proliferation of human cells relevant to tissue reconstruction, was determined as follows.

Scaffolds were fabricated using 2% w:v fibrinogen and 5% PVA, both dissolved in MES, 0.15 M NaCl, pH 7.40 buffer, in a final concentration of 0.5% fibrinogen, PVA, together with 0.4 IU/mg IU Thrombin.

Scaffolds were subsequently cross-linked with either glutaraldehyde (GLA) or EDC/NHS. Both of these cross-linking reactions were carried out in 80% ethanol buffered to pH 7.4 with 0.1 M MES_{(aq)}. For glutaraldehyde, cross-linking was performed at 2 concentrations, 0.05% or 0.2%, for 4 h. The scaffolds were washed 5 times for 15 min in 0.1% sodium borohydride to reduce the glutaraldehyde adducts.

For 1-ethyl-3-(-3-dimethylaminopropyl) carbodiimide hydrochloride (EDC) and sulfo-N-hydroxysuccinimide (NHS), cross-linking with (21.9:8.68 mM, 5:2 EDC:NHS molar ratio) for 30 m or 2 h.

After cross-linking step, scaffolds were washed 5 times for 15 minutes in deionised water and then lyophilised at −40° C.

Scaffolds (14 mm diameter samples) in 14 mm diameter well tissue culture plates were washed twice in Dulbecco's PBS without Calcium and Magnesium salts, equilibrated in tissue culture medium (Dulbecco-modified Eagles Minimal medium supplemented with 10% foetal bovine serum, 0.4 mM glutamax, 100 U/ml penicillin and 100 ug/ml streptomycin) and seeded with cells (h-tert BM-MSC), in culture medium (1×10⁵/well), and transferred after 24 hours to a fresh culture well. Culture medium was changed every 2 days. Cell number was determined by incubating cultures in CCK-8 reagent (WST-8 [2-(2-methoxy-4-nitrophenyl)-3-(4-nitrophenyl)-5-(2,4-disulfophenyl)-2H-tetrazolium, monosodium salt]) for 3 hours, and measuring the optical absorbance (A₄₅₀) of a sample of supernatant against the same reagent in culture medium unexposed to culture. The assay was calibrated against a dilution series of measured cell numbers, after overnight seeding into tissue culture wells.

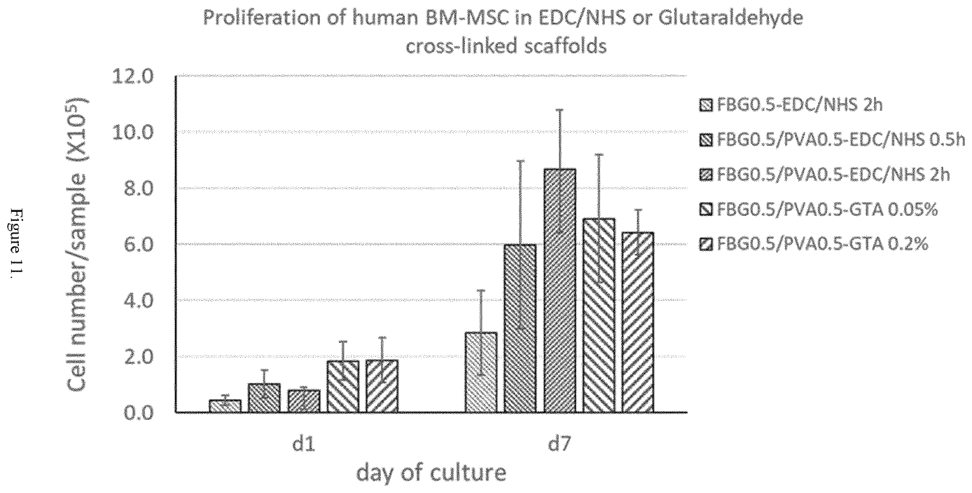

The result (shown in FIG. 11) demonstrated proliferation of cells occurred over a 7 day period in each scaffold, with some differences between cross-linking regimes as shown.

1. A method for manufacturing a protein-based three-dimensional tissue scaffold, the method comprising the steps of:
(a) providing at least one protein capable of triggered polymerization;
(b) forming a high-internal phase oil/water emulsion comprising the protein(s) of step (a) in aqueous phase, further comprising a surfactant which supports a high-internal phase emulsion regime;
(c) triggering polymerization of the at least one protein comprised in the high-internal phase emulsion of step (b); and
(d) washing out the oil phase.

2. The method of claim 1, wherein the at least one protein capable of triggered polymerization of step (a) is selected from fibrinogen, collagen, laminin, elastin, cultured cell extracellular matrix extracts, and extracellular matrix preparations from natural tissue.

3. The method of claim 2, wherein the at least one protein capable of triggering polymerization of step (a) is fibrinogen, and wherein step (c) is performed by addition of thrombin to the emulsion of step (b).

4. The method of claim 1,
wherein the surfactant which supports the high-internal phase emulsion regime of step (b) comprises:
at least one optionally substituted polyethylene glycol chain comprising 5 to 16 ether bonds, and
at least one aliphatic hydrocarbon chain of at least 8 carbon atoms, wherein at least one tertiary or quaternary carbon atom is present in the at least one aliphatic hydrocarbon chain.

5. The method of claim 1, wherein the surfactant which supports the high-internal phase emulsion regime of step (b) exhibits oil-carrying capacity (OCC) in the range from 75% to 95% and preferably hydrophilic-lipophilic balance (HLB) in the range from 12 to 16.

6. The method of claim 1, wherein the surfactant which supports the high-internal phase emulsion regime of step (b) is selected from tergitol-NP10, Tergitol-TMN-6; Tergitol-TMN-10, triton-X100-R, Triton X104R, Triton X114R, Triton-CG110, Triton X165, and Ecosurf-EH-9.

7. The method of claim 1, further comprising a step of crosslinking the protein-based three-dimensional tissue scaffold obtained in step (d).

8. The method of claim 1, further comprising a step of freeze-drying the protein-based three-dimensional tissue scaffold.

9. A protein-based three-dimensional tissue scaffold obtainable according to the method of claim 1.

10. A protein-based three-dimensional tissue scaffold comprising polymerized protein(s) of porosity of at least 74%.

11. The protein-based three-dimensional tissue scaffold of claim 10, wherein the polymerized protein(s) comprise(s) fibrin.

12. The protein-based three-dimensional tissue scaffold of claim 9, further comprising a non-polypeptide polymer, preferably a polyvinyl alcohol.

13. A method of producing a cell culture substrate, comprising a step wherein the protein-based three-dimensional tissue scaffold of claim 9 is deposited using additive manufacturing techniques.

14. Use of the protein-based three-dimensional tissue scaffold of claim 9 in an in vitro tissue culture.

15. Use of the protein-based three-dimensional tissue scaffold of claim 9 in in vitro tissue engineering.

16. Use of claim 15, wherein in vitro tissue engineering comprises preparation of a myocardial patch.

17. The protein-based three-dimensional tissue scaffold of claim 9 for use in therapy.

18. The protein-based three-dimensional tissue scaffold of claim 9 for use in tissue reconstruction.

19. The protein-based three dimensional tissue scaffold for use of claim 18, wherein the tissue reconstruction is a dermal replacement or substitute.

20. The protein-based three dimensional tissue scaffold for use of claim 18, wherein the tissue reconstruction is used in the treatment of a wound.

21. The protein-based three dimensional tissue scaffold for use of claim 20, wherein the wound is selected from a skin-loss wound, a chronic wound, a non-healing wound, a third degree burn, a fourth degree tissue loss, a traumatic skin-stripping wound and a surgical excision wound.

22. The protein-based three dimensional tissue scaffold for use of claim 18, wherein the protein-based three dimensional tissue scaffold is positioned on the wound bed as acellular material.
